(19) United States
(12) Patent Application Publication
(10) Pub. No.: US 2025/0248813 A1
(43) Pub. Date: Aug. 7, 2025

(54) Loader Sheath and Methods for Preparing Prosthetic Heart Valve Delivery System

(71) Applicant: St. Jude Medical, Cardiology Division, Inc., St. Paul, MN (US)

(72) Inventors: Tyler Govek, St. Louis Park, MN (US); Neil Theisen, Champlin, MN (US)

(73) Assignee: St. Jude Medical, Cardiology Division, Inc., St. Paul, MN (US)

(21) Appl. No.: 18/943,056

(22) Filed: Nov. 11, 2024

Related U.S. Application Data
(60) Provisional application No. 63/548,895, filed on Feb. 2, 2024.

(51) Int. Cl. A61F 2/24

(52) U.S. Cl. CPC A61F 2/2433; A61F 2/2436

(57) ABSTRACT

A prosthetic heart valve delivery system may include a delivery device having a balloon at a distal end thereof, a balloon-expandable prosthetic heart valve configured to be received on the balloon in a crimped condition, and a loader sheath configured to at least partially surround the crimped prosthetic heart valve while the crimped prosthetic heart valve is received on the balloon. The loader sheath may include a tube and a proximal hub. The loader sheath may include a first channel extending in an axial direction along a wall of the tube and a second channel extending parallel to the first channel. The first channel and the second channel may form weakened areas to promote tearing of the tube along the first channel and along the second channel.

### CROSS-REFERENCE TO RELATED APPLICATIONS

This application claims priority to the filing date of U.S. Provisional Patent Application No. 63/548,895, filed Feb. 2, 2024, the disclosure of which is hereby incorporated by reference herein.

### BACKGROUND OF THE DISCLOSURE

Valvular heart disease, and specifically aortic and mitral valve disease, is a significant health issue in the United States. Valve replacement is one option for treating heart valve diseases. Prosthetic heart valves include surgical heart valves, as well as collapsible and expandable heart valves intended for transcatheter aortic valve replacement or implantation (“TAVR” or “TAVI”) or transcatheter mitral valve replacement (“TMVR”). Surgical or mechanical heart valves may be sutured into a native annulus of a patient during an open-heart surgical procedure, for example. Collapsible and expandable heart valves may be delivered into a patient via a delivery apparatus such as a catheter to avoid a more invasive procedure such as full open-chest, open-heart surgery. As used herein, reference to a “collapsible and expandable” heart valve includes heart valves that are formed with a small cross-section that enables them to be delivered into a patient through a catheter in a minimally invasive procedure, and then expanded to an operable state once in place, as well as heart valves that, after construction, are first collapsed to a small cross-section for delivery into a patient and then expanded to an operable size once in place in the valve annulus.

The present disclosure addresses problems and limitations associated with the related art.

### SUMMARY OF THE DISCLOSURE

According to one example of the disclosure, a prosthetic heart valve delivery system may include a delivery device having a balloon at a distal end thereof, a balloon-expandable prosthetic heart valve configured to be received on the balloon in a crimped condition, and a loader sheath configured to at least partially surround the crimped prosthetic heart valve while the crimped prosthetic heart valve is received on the balloon. The loader sheath may include a tube and a proximal hub. The loader sheath may include a first channel extending in an axial direction along a wall of the tube and a second channel extending parallel to the first channel. The first channel and the second channel may form weakened areas to promote tearing of the tube along the first channel and along the second channel. The tube may have an outer diameter and an inner diameter, and the first channel and the second channel may be formed in a wall of the tube such that the first channel and second channel open to the inner diameter of the tube. The tube may have an outer diameter and an inner diameter, and the first channel and the second channel may be formed in a wall of the tube such that the first channel and second channel open to the outer diameter of the tube. The tube may have an outer diameter and an inner diameter, and the first channel and the second channel may be formed entirely within a wall thickness of the tube so that the first channel and the second channel are each enclosed within the wall thickness of the tube. The tube may be overmolded on the proximal hub. The tube may be formed of fluorinated ethylene propylene (“FEP”), polytetrafluoroethylene (“PTFE”), nylon, or high density polyethylene (“HDPE”). The first channel and the second channel may be formed of a first polymer, and portions of the tube excluding the first channel and the second channel may be formed of a second polymer, the second polymer having a rigidity that is greater than a rigidity of the first polymer. A pull tab may be fixed to the tube, and the pull tab may directly connect to the tube at locations of the first channel and the second channel.

According to another example of the disclosure, a method of preparing a delivery system for an implantation includes coupling a fluid reservoir to a delivery device of the delivery system so that the fluid reservoir is in fluid communication with a balloon of the delivery device. While a balloon-expandable prosthetic heart valve is crimped over the balloon, and while a loader sheath at least partially surrounds the crimped prosthetic heart valve and the balloon, the balloon may be pressurized by pressurizing the fluid reservoir. When pressurizing the balloon, the loader sheath may limit an amount which the balloon and the prosthetic heart valve may expand. Pressurizing the balloon may cause at least one air bubble within the balloon to compress. After pressurizing the balloon, the fluid reservoir may be depressurized to withdraw the at least one air bubble from the balloon.

According to a further example of the disclosure, a method of implanting a prosthetic heart valve into a patient may include receiving a delivery system contained within packaging, the delivery system including (i) a delivery device having a balloon at a distal end thereof, (ii) a balloon-expandable prosthetic heart valve crimped over the balloon, and (iii) a loader sheath at least partially surrounding the crimped prosthetic heart valve and the balloon. The delivery system may be removed from the packaging, and an introducer may be inserted at least partially into vasculature of the patient. The delivery system may be advanced at least partially into the introducer until the loader sheath passes through a hemostasis valve of the introducer. The delivery system may be advanced so that the prosthetic heart valve exits the loader sheath, passes through the introducer, and is positioned within a native valve annulus. The prosthetic heart valve may be deployed into the native valve annulus by inflating the balloon.

According to still another example of the disclosure, a prosthetic heart valve delivery system may include a delivery device having a balloon at a distal end thereof, and a balloon-expandable prosthetic heart valve configured to be received on the balloon in a crimped condition. An introducer may have an introducer hub and an introducer sheath extending distally from the introducer hub, and a first hemostasis valve may be positioned within the introducer hub. A loader sheath may be configured to at least partially surround the crimped prosthetic heart valve while the crimped prosthetic heart valve is received on the balloon, and the loader sheath may include a tube sized to be received within the introducer hub. The introducer may include a first coupler and the loader sheath may include a second coupler, and the first coupler may be configured to couple to the second coupler when the tube of the loader sheath is received within introducer hub. The first coupler and the second coupler may each be threads. The first coupler and the second coupler may include a latch, hook, or detent. When the first coupler is coupled to the second coupler, the tube of the loader may include a distal length received within the introducer hub and a proximal length extending out of the introducer hub. When the first coupler is coupled to the second coupler, the distal length and the proximal length may each be about half of a total length of the tube. When the first coupler is coupled to the second coupler, the distal length may be about 75% of a total length of the tube and the proximal length may be about 25% of the total length of the tube. When the first coupler is coupled to the second coupler, the distal length may be about 25% of a total length of the tube and the proximal length may be about 75% of the total length of the tube.

According to yet another example of the disclosure, a method of implanting a prosthetic heart valve into a patient includes inserting an introducer at least partially into vasculature of the patient, and inserting a distal end of a delivery device into the introducer while a balloon-expandable prosthetic heart valve is crimped over a balloon of the delivery device and while a loader sheath at least partially surrounds the crimped prosthetic heart valve and the balloon. The delivery device may be advanced into the introducer until a tube of the loader sheath passes through a first hemostasis valve of the introducer. The tube of the loader sheath may be coupled to a hub of the introducer so that the loader sheath and the introducer become fixed together. The delivery system may be advanced so that the prosthetic heart valve exits the loader sheath, passes through the introducer, and is positioned within a native valve annulus. The prosthetic heart valve may be deployed into the native valve annulus by inflating the balloon. Coupling the tube of the loader sheath to the hub of the introducer may include threading the tube with the hub of the introducer. Coupling the tube of the loader sheath to the hub of the introducer may include latching or hooking the tube onto the hub of the introducer. During the step of advancing the delivery system so that the prosthetic heart valve exits the loader sheath, passes through the introducer, and is positioned within a native valve annulus, the tube of the loader sheath may remain coupled to the hub of the introducer. During the step of deploying the prosthetic heart valve into the native valve annulus by inflating the balloon, the tube of the loader sheath may remain coupled to the hub of the introducer. The loader sheath may include a second hemostasis valve. After coupling the tube of the loader sheath to the hub of the introducer, the tube of the loader may include a distal length received within the hub of the introducer and a proximal length extending out of the hub of the introducer. After coupling the tube of the loader sheath to the hub of the introducer, the distal length and the proximal length may each about half of a total length of the tube. After coupling the tube of the loader sheath to the hub of the introducer, the distal length may be about 75% of a total length of the tube and the proximal length may be about 25% of the total length of the tube. After coupling the tube of the loader sheath to the hub of the introducer, the distal length may be about 25% of a total length of the tube and the proximal length may be about 75% of the total length of the tube.

According to still another example of the disclosure, a prosthetic heart valve delivery system includes a delivery device having a balloon at a distal end thereof, a balloon-expandable prosthetic heart valve configured to be received on the balloon in a crimped condition, and a first loader sheath tube configured to at least partially surround the crimped prosthetic heart valve while the crimped prosthetic heart valve is received on the balloon. The first loader sheath tube may have a first inner diameter and may be configured to reversibly couple to a proximal loader sheath hub. A second loader sheath tube may be configured to at least partially surround the crimped prosthetic heart valve while the crimped prosthetic heart valve is received on the balloon. The second loader sheath tube may have a second inner diameter smaller than the first inner diameter. The second loader sheath tube may be configured to reversibly couple to the proximal loader sheath hub. The system may have a packaged configuration in which the prosthetic heart valve delivery system is stored within packaging, the first loader sheath tube (i) being coupled to the proximal loader sheath hub and (ii) at least partially surrounding the crimped prosthetic heart valve while the crimped prosthetic heart valve is received on the balloon in the packaged configuration so that the prosthetic heart valve has a first outer crimped diameter about equal to the first inner diameter. The system may have a use configuration in which the prosthetic heart valve delivery system is not stored within the packaging, the second loader sheath tube (i) being coupled to the proximal loader sheath hub and (ii) at least partially surrounding the crimped prosthetic heart valve while the crimped prosthetic heart valve is received on the balloon in the use configuration so that the prosthetic heart valve has a second outer crimped diameter about equal to the second inner diameter.

According to another aspect of the disclosure, a method of implanting a prosthetic heart valve into a patient includes receiving a delivery system contained within packaging, the delivery system including (i) a delivery device having a balloon at a distal end thereof, (ii) a balloon-expandable prosthetic heart valve crimped over the balloon, and (iii) a first loader tube at least partially surrounding the crimped prosthetic heart valve and the balloon so that the crimped prosthetic heart valve has a first crimped outer diameter. The delivery system may be removed from the packaging. The first loader tube may be decoupled from a proximal loader sheath hub. The first loader tube may be removed from the delivery device so that the first loader tube does not at least partially surround the crimped prosthetic heart valve. After removing the first loader tube, the prosthetic heart valve may be further crimped over the balloon so that the crimped prosthetic heart valve has a second crimped outer diameter smaller than the first crimped outer diameter. After further crimping the prosthetic heart valve, a second loader tube may be coupled to the proximal loader sheath hub so that the second loader tube at least partially surrounds the crimped prosthetic heart valve, the second loader tube having a second inner diameter that is smaller than a first inner diameter of the first loader tube. The balloon-expandable prosthetic heart valve may be deployed within a native valve annulus of the patient. The method may further include inserting an introducer into vasculature of the patient, advancing the delivery device into the introducer until the second loader tube passes through a hemostasis valve of the introducer, advancing the delivery device so that the prosthetic heart valve exits the second loader tube, passes through the introducer, and is positioned within the native valve annulus, and inflating the balloon to deploy the balloon-expandable prosthetic heart valve. Between (i) removing the delivery system from the packaging and (ii) deploying the balloon-expandable prosthetic heart valve within the native valve annulus of the patient, the first loader tube may not be inserted into the introducer.

According to still another aspect of the disclosure, a prosthetic heart valve delivery system includes a delivery device having a balloon at a distal end thereof, a balloon-expandable prosthetic heart valve configured to be received on the balloon in a crimped condition, and a loader sheath including a tube configured to at least partially surround the crimped prosthetic heart valve while the crimped prosthetic heart valve is received on the balloon. The tube may have an intermediate section formed of a semi-compliant material so that, when the tube at least partially surrounds the crimped prosthetic heart valve, a crimping force on the intermediate section can be transferred to the crimped prosthetic heart valve to further crimp the prosthetic heart valve. The semi-compliant material may be a low durometer polyether block amide. The tube may have a length, and an entirety of the length of the tube may be formed of the semi-compliant material. The tube may have a length, and at least part of the length of the tube proximal to the intermediate section or distal to the intermediate section may be formed of a non-compliant material. The non-compliant material may be fluorinated ethylene propylene (“FEP”), polytetrafluoroethylene (“PTFE”), nylon, or high density polyethylene (“HDPE”).

According to another example of the disclosure, a method of implanting a prosthetic heart valve into a patient includes receiving a delivery system contained within packaging, the delivery system including (i) a delivery device having a balloon at a distal end thereof, (ii) a balloon-expandable prosthetic heart valve crimped over the balloon, and (iii) a loader tube at least partially surrounding the crimped prosthetic heart valve and the balloon so that the crimped prosthetic heart valve has a first crimped outer diameter, The delivery system may be removed from the packaging. A crimper may be placed over the loader tube and a crimping force may be applied onto the loader tube to further crimp the prosthetic heart valve to have a second crimped outer diameter that is smaller than the first crimped outer diameter. The balloon-expandable prosthetic heart valve may be deployed within a native valve annulus of the patient. The tube may have an intermediate section formed of a semi-compliant material. During applying the crimping force onto the loader tube, the intermediate section of the loader tube may be aligned with the prosthetic heart valve. The semi-compliant material may be a low durometer polyether block amide. The tube may have a length, and an entirety of the length of the tube may be formed of the semi-compliant material. The tube may have a length, and at least part of the length of the tube proximal to the intermediate section or distal to the intermediate section may be formed of a non-compliant material. The non-compliant material may be fluorinated ethylene propylene (“FEP”), polytetrafluoroethylene (“PTFE”), nylon, or high density polyethylene (“HDPE”). The method may further include inserting an introducer into vasculature of the patient, advancing the delivery device into the introducer until the loader tube passes through a hemostasis valve of the introducer, advancing the delivery device so that the prosthetic heart valve exits the loader tube, passes through the introducer, and is positioned within the native valve annulus, and inflating the balloon to deploy the balloon-expandable prosthetic heart valve.

According to another aspect of the disclosure, a prosthetic heart valve delivery system may include a delivery device having a balloon at a distal end thereof, a balloon-expandable prosthetic heart valve configured to be received on the balloon in a crimped condition, and a loader sheath including a tube configured to at least partially surround the crimped prosthetic heart valve while the crimped prosthetic heart valve is received on the balloon. The tube may have a proximal section with a first inner diameter, and a distal section that tapers from the first inner diameter to a second inner diameter that is smaller than the first inner diameter. The system may have a packaged configuration in which the prosthetic heart valve delivery system is stored within packaging and the prosthetic heart valve is positioned solely within the proximal section of the tube.

According to a further aspect of the disclosure, a method of implanting a prosthetic heart valve into a patient may include receiving a delivery system contained within packaging, the delivery system including (i) a delivery device having a balloon at a distal end thereof, (ii) a balloon-expandable prosthetic heart valve crimped over the balloon, and (iii) a loader tube at least partially surrounding the crimped prosthetic heart valve and the balloon so that the crimped prosthetic heart valve has a first crimped outer diameter. The delivery system may be removed from the packaging. An introducer may be inserted into vasculature of the patient. The delivery device may be advanced into the introducer until the loader tube passes through a hemostasis valve of the introducer. The delivery device may be advanced so that the prosthetic heart valve advances through the loader tube, passes through the introducer, and is positioned within the native valve annulus. The balloon-expandable prosthetic heart valve may be deployed within a native valve annulus of the patient. During advancing the delivery device so that the prosthetic heart valve advances through the loader tube, the prosthetic heart valve may crimp to a second crimped outer diameter that is smaller than the first crimped outer diameter. The tube may have a proximal section with a first inner diameter, and a distal section that tapers from the first inner diameter to a second inner diameter that is smaller than the first inner diameter. Prior to removing the delivery system from the packaging, the prosthetic heart valve may be positioned solely within the proximal section of the tube.

According to a further example of the disclosure, a method of preparing a prosthetic heart valve delivery system for use may include positioning a balloon-expandable prosthetic heart valve over a balloon while the prosthetic heart valve has an uncrimped outer diameter, the balloon being positioned at a distal end of a delivery device of the prosthetic heart valve delivery system. A fluid reservoir may be coupled to the delivery device so that the fluid reservoir is in fluid communication with the balloon. The fluid reservoir may be depressurized to depressurize the balloon. While the balloon is depressurized, the prosthetic heart valve may be crimped over the balloon so that the prosthetic heart valve has a crimped outer diameter that is smaller than the uncrimped outer diameter. A tube of a loader sheath may be positioned over the crimped prosthetic heart valve so that the loader sheath at least partially surrounds the crimped prosthetic heart valve. During the step of positioning the tube of the loader sheath over the crimped prosthetic heart valve, the balloon may remain depressurized. The fluid reservoir may be a syringe, and depressurizing the syringe may include retracting a plunger handle of the syringe. The plunger handle may be released after positioning the tube of the loader sheath over the crimped prosthetic heart valve.

According to another examples of the disclosure, a method of implanting a prosthetic heart valve into a patient may include coupling a fluid reservoir to a delivery device so that the fluid reservoir is in fluid communication with a balloon at a distal end of the delivery device. An introducer may be inserted into vasculature of the patient. The delivery device may be advanced into the introducer while a balloon-expandable prosthetic heart valve is crimped over the balloon. The delivery device may be further advanced so that the prosthetic heart valve advances through the introducer, and is positioned within a native valve annulus. The prosthetic heart valve may be deployed within the native valve annulus. In a first stage prior to advancing the delivery device into the introducer, the prosthetic heart valve may be crimped over a central portion of the balloon so that the prosthetic heart valve has a crimped outer diameter, a proximal portion of the balloon proximal to the central portion of the balloon has a proximal outer diameter, and a distal portion of the balloon distal to the central portion of the balloon has a distal outer diameter. The proximal outer diameter and the distal outer diameter may both be larger than the crimped outer diameter. In a second stage after the first stage but prior to advancing the delivery device into the introducer, the fluid reservoir may be depressurized to depressurize the balloon to reduce a size of the proximal outer diameter and a size of the distal outer diameter. In the first stage, the proximal outer diameter may be about the same as the distal outer diameter. In the first stage, the proximal outer diameter and the distal outer diameter may both be between about 8 mm and about 12 mm. In the first stage, the crimped outer diameter may be between about 6 mm and about 10 mm. In the second stage, the proximal outer diameter may be about the same as the distal outer diameter and the crimped outer diameter. After the prosthetic heart valve advances through the introducer, but before the prosthetic heart valve is positioned within the native valve annulus, the fluid reservoir may be repressurized to repressurize the balloon to increase the size of the proximal outer diameter and the size of the distal outer diameter. Repressurizing the fluid reservoir may be performed while the prosthetic heart valve is within a descending aorta of the patient. The fluid reservoir may be a syringe, and depressurizing the syringe may include retracting a plunger handle of the syringe. Repressurizing the fluid reservoir may include advancing the plunger handle of the syringe.

According to still another example of the disclosure, a prosthetic heart valve delivery system may include a delivery device having a balloon at a distal end thereof, the delivery device including a steering catheter, an outer balloon catheter received within the steering catheter, and a first lumen positioned between the outer balloon catheter and the steering catheter. They system may also include a balloon-expandable prosthetic heart valve, and a loader sheath. In an assembled condition of the prosthetic heart valve delivery system, (i) the prosthetic heart valve may be crimped over the balloon, (ii) the tube of the loader sheath may at least partially surround the crimped prosthetic heart valve, and (iii) a distal end of the first lumen may be contiguous with an interior space of the loader sheath. The delivery device may include a handle and a flush port coupled to the handle. The flush port may be in fluid communication with the first lumen.

According to still a further example of the disclosure, a method of preparing a prosthetic heart valve delivery system for use includes coupling a fluid reservoir to a flush port on a handle of a delivery device of the delivery system, the delivery device including a steering catheter, an outer balloon catheter received within the steering catheter, and a first lumen positioned between the outer balloon catheter and the steering catheter. Fluid may be advanced through the flush port while the delivery system is in an assembled condition, wherein in the assembled condition of the delivery system, (i) a prosthetic heart valve is crimped over a balloon at a distal end of the delivery device, (ii) a tube of a loader sheath at least partially surrounds the crimped prosthetic heart valve, and (iii) a distal end of the first lumen is contiguous with an interior space of the loader sheath. The fluid that advances through the flush port on the handle may also advance through the first lumen and into the interior space of the loader sheath. The fluid reservoir may be a syringe. The fluid may include saline.

### BRIEF DESCRIPTION OF THE DRAWINGS

FIG. 1 is a perspective view of an example of a prosthetic heart valve.

FIG. 2 is a front view of an example of a section of the frame of the prosthetic heart valve of FIG. 1, as if cut longitudinally and laid flat on a table.

FIG. 3 is a front view of an example of a prosthetic leaflet of the prosthetic heart valve of FIG. 1, as if laid flat on a table.

FIG. 4 is a top view of the prosthetic heart valve of FIG. 1 mounted on an example of a portion of a delivery system.

FIG. 5 is an enlarged view of the handle of the delivery system shown in FIG. 4.

FIG. 6 is an enlarged view of a distal end of the delivery system shown in FIG. 4.

FIG. 7 is a top view of an example of a balloon catheter when the balloon is inflated.

FIG. 8 is a top view of an example of an inflation system for use with a delivery system similar to that shown in FIG. 4.

FIG. 9 is a side view of the inflation system of FIG. 8.

FIG. 10 is a perspective view of a connection between the inflation system of FIGS. 8-9 and the handle of the delivery system of FIG. 4.

FIG. 11 is a flowchart showing exemplary steps in a procedure to implant the prosthetic heart valve of FIG. 1 into a patient using the delivery system of FIG. 4.

FIG. 12A is a cross-section of an example of the distal end of the delivery system of FIG. 4 with an example of loader sheath assembled thereto.

FIG. 12B is a highly schematic view of an example of the distal end of the delivery system of FIG. 4 with an air bubble trapped therein.

FIG. 12C is a highly schematic view of an example of a loader sheath assembled to the

distal end of the delivery system shown in FIG. 12B while the delivery system is pressurized.

FIG. 12D is a cross-section of an example of a loader sheath coupled to an example of the introducer of the delivery system of FIG. 4.

FIGS. 12E-12H are highly schematic transverse cross-sections of examples of tubes of a loader sheath.

FIGS. 12I-12J are highly schematic views of an example of a loader tube prior to and after, respectively, performing a successful peel-away operation.

FIG. 12K is a highly schematic view of an example of a loader tube after an attempted peel-away operation has failed.

FIG. 12L is a side view of an example of a loader sheath.

FIG. 12M is a longitudinal cross-section of the loader sheath of FIG. 12L assembled over an example of a delivery device.

FIG. 13A is a highly schematic longitudinal cross-section of an example of a relatively large diameter tube of a first loader sheath assembled to a crimped prosthetic heart valve.

FIG. 13B is a highly schematic longitudinal cross-section of an example of a relatively large diameter tube of a second loader sheath assembled to the crimped prosthetic heart valve of FIG. 13A.

FIG. 14 is a highly schematic longitudinal cross-section of an example of a loader sheath with a compliant section assembled to a crimped prosthetic heart valve.

FIG. 15 is a highly schematic longitudinal cross-section of an example of a tapered loader sheath assembled to a crimped prosthetic heart valve.

FIG. 16A is a highly schematic transverse cross-section of an example of a prosthetic heart valve crimped over a balloon, the prosthetic heart valve having a crimp profile.

FIG. 16B is a highly schematic transverse cross-section of an example of a prosthetic heart valve crimped over a balloon under a vacuum, the prosthetic heart valve having a crimp profile smaller than the crimp profile of FIG. 16A.

FIG. 17A is a highly schematic longitudinal cross-section of an example of a prosthetic

heart valve crimped over a balloon of a delivery device prior to being inserted into a patient.

FIG. 17B is a highly schematic longitudinal cross-section of the example of FIG. 17A after a vacuum has been pulled in the balloon.

FIG. 17C is a highly schematic longitudinal cross-section of the example of FIG. 17B after the vacuum has been released.

FIG. 18 is a highly schematic view of an example of continuous flush lumen extending from a delivery device handle, through an interior of a steering catheter, and into an interior space of a loader sheath.

FIG. 19A is a flowchart of an example of a first stage of a method of preparing a delivery system for implanting a prosthetic heart valve.

FIG. 19B is a flowchart of an example of a second stage of a method of preparing a delivery system for implanting a prosthetic heart valve.

FIG. 19C is a flowchart of an example of a method of delivering a prosthetic heart valve into a patient.

### DETAILED DESCRIPTION OF THE DISCLOSURE

As used herein, the term “inflow end” when used in connection with a prosthetic heart valve refers to the end of the prosthetic valve into which blood first enters when the prosthetic valve is implanted in an intended position and orientation, while the term “outflow end” refers to the end of the prosthetic valve where blood exits when the prosthetic valve is implanted in the intended position and orientation. Thus, for a prosthetic aortic valve, the inflow end is the end nearer the left ventricle while the outflow end is the end nearer the aorta. The intended position and orientation are used for the convenience of describing valves disclosed herein. However, it should be noted that the use of the valve is not limited to the intended position and orientation but may be deployed in any type of lumen or passageway. For example, although prosthetic heart valves are described herein as prosthetic aortic valves, those same or similar structures and features can be employed in other heart valves, such as the pulmonary valve, the mitral valve, or the tricuspid valve. Further, the term “proximal,” when used in connection with a delivery device or system, refers to a position relatively close to the user of that device or system when it is being used as intended, while the term “distal” refers to a position relatively far from the user of the device. In other words, the leading end of a delivery device or system is positioned distal to the trailing end of the delivery device or system, when the delivery device is being used as intended. As used herein, the terms “substantially,” “generally,” “approximately,” and “about” are intended to mean that slight deviations from absolute are included within the scope of the term so modified. As used herein, the prosthetic heart valves may assume an “expanded state” and a “collapsed state,” which refer to the relative radial size of the stent.

Collapsible and expandable prosthetic heart valves typically take the form of a one-way valve structure (often referred to as a valve assembly) mounted within an expandable frame (the terms “stent” and “frame” may be used interchangeably herein). In general, these collapsible and expandable heart valves include a self-expanding, mechanically-expandable, or balloon-expandable frame, often made of nitinol or another shape-memory metal or metal alloy (for self-expanding frames) or steel or cobalt chromium (for balloon-expandable frames). The one-way valve assembly mounted to/within the stent includes one or more leaflets and may also include a cuff or skirt. The cuff may be disposed on the stent's interior or luminal surface, its exterior or abluminal surface, and/or on both surfaces. A cuff helps to ensure that blood does not just flow around the valve leaflets if the valve or valve assembly is not optimally seated in a valve annulus. A cuff, or a portion of a cuff disposed on the exterior of the stent, can help prevent leakage around the outside of the valve (the latter known as paravalvular or “PV” leakage).

Balloon expandable valves are typically delivered to the native annulus while collapsed (or “crimped”) onto a deflated balloon of a balloon catheter, with the collapsed valve being either covered or uncovered by an overlying sheath. Once the crimped prosthetic heart valve is positioned within the annulus of the native heart valve that is being replaced, the balloon is inflated to force the balloon-expandable valve to transition from the collapsed or crimped condition into an expanded or deployed condition, with the prosthetic heart valve tending to remain in the shape into which it is expanded by the balloon. Typically, when the position of the collapsed prosthetic heart valve is determined to be in the desired position relative to the native annulus (e.g. via visualization under fluoroscopy), a fluid (typically a liquid although gas could be used as well) such as saline is pushed via a syringe (manually, automatically, or semi-automatically) through the balloon catheter to cause the balloon to begin to fill and expand, and thus cause the overlying prosthetic heart valve to expand into the native annulus.

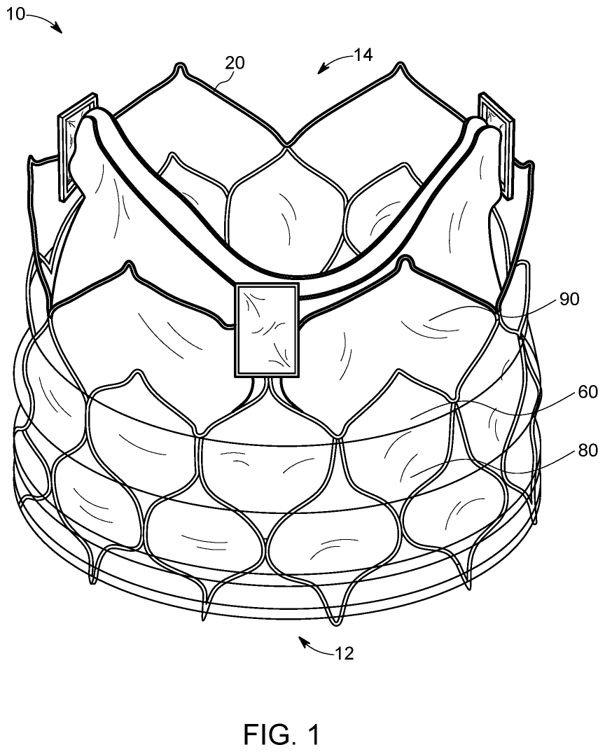

FIG. 1 is a perspective view of one example of a prosthetic heart valve 10. Prosthetic heart valve 10 may be a balloon-expandable prosthetic aortic valve, although in other examples it may be a self-expandable or mechanically-expandable prosthetic heart valve, intended for replacing a native aortic valve or another native heart valve. Prosthetic heart valve 10 is shown in an expanded condition in FIG. 1. Prosthetic heart valve 10 may extend between an inflow end 12 and an outflow end 14. Prosthetic heart valve 10 may include a collapsible and expandable frame 20, an inner cuff or skirt 60, an outer cuff or skirt 80, and a plurality of prosthetic leaflets 90. As should be clear below, prosthetic heart valve 10 is merely one example of a prosthetic heart valve, and other examples of prosthetic heart valves may be suitable for use with the concepts described below.

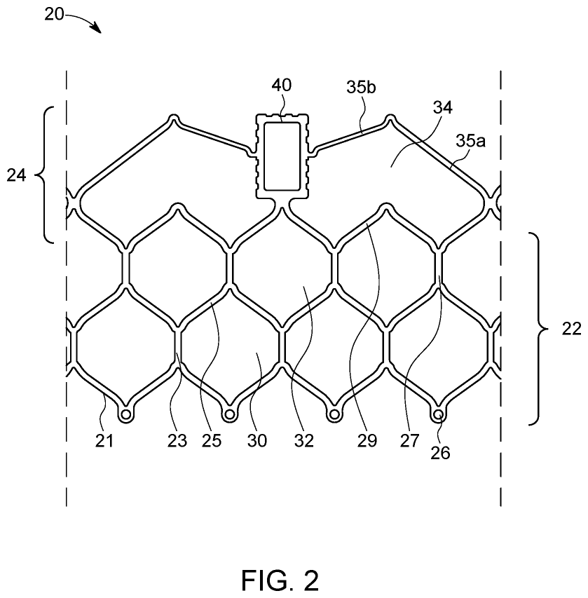

FIG. 2 is a front view of an example of a section of the frame 20 of prosthetic heart valve 10, as if cut longitudinally and laid flat on a table. The section of frame 20 in FIG. 2 may represent approximately one-third of a complete frame, particularly if frame 20 is used in conjunction with a three-leaflet prosthetic heart valve. In the illustrated example, frame 20 is a balloon-expandable stent and may be formed of stainless steel or cobalt-chromium, and which may include additional materials such as nickel and/or molybdenum. However, in some embodiments the stent may be formed of a shape memory material such as nitinol or the like. The frame 20, when provided as a balloon-expandable frame, is configured to collapse upon being crimped to a smaller diameter and/or expand upon being forced open, for example via a balloon within the frame expanding, and the frame will substantially maintain the shape to which it is modified when at rest.

Frame 20 may include an inflow section 22 and an outflow section 24. The inflow section 22 may also be referred to as the annulus section. In one example, the inflow section 22 includes a plurality of rows of generally hexagon-shaped cells. For example, the inflow section 22 may include an inflow-most row of hexagon-shaped cells 30 and an outflow-most row of hexagon-shaped cells 32. The inflow-most row of hexagonal cells 30 may be formed of a first circumferential row of angled or zig-zag struts 21, a second circumferential row of angled or zig-zag struts 25, and a plurality of axial struts 23 that connect the two rows. In other words, each inflow-most hexagonal cell 30 may be formed by two angled struts 21 that form an apex pointing in the inflow direction, two angled struts 25 that form an apex pointing in the outflow direction, and two axial struts that connect the two angled struts 21 to two corresponding angled struts 25. The outflow-most row of hexagonal cells 32 may be formed of the second circumferential row of angled or zig-zag struts 25, a third circumferential row of angled or zig-zag struts 29, and a plurality of axial struts 27 that connect the two rows. In other words, each outflow-most hexagonal cell 32 may be formed by two angled struts 25 that form an apex pointing in the inflow direction, two angled struts 29 that form an apex pointing in the outflow direction, and two axial struts that connect the two angled struts 27 to two corresponding angled struts 29. It should be understood that although the term “outflow-most” is used in connection with hexagonal cells 32, additional frame structure, described in more detail below, is still provided in the outflow direction relative to the outflow-most row of hexagonal cells 32.

In the illustrated embodiment, assuming that frame 20 is for use with a three-leaflet valve and thus the section shown in FIG. 2 represents about one-third of the frame 20, each row of cells 30, 32 includes twelve individual cells. However, it should be understood that more or fewer than twelve cells may be provided per row of cells. Further, the inflow or annulus section 22 may include more or fewer than two rows of cells. Still further, although cells 30, 32 are shown as being hexagonal, the some or all of the cells of the inflow section 22 may have other shapes, such as diamond-shaped, chevron-shaped, or other suitable shapes. In the illustrated embodiment, every cell 30 in the first row is structurally similar or identical to every other cell 30 in the first row, every cell 32 in the second row is structurally similar or identical to every other cell 32 in the second row, and every cell 30 in the first row is structurally similar or identical (excluding the aperture 26) to every cell 32 in the second row. However, in other examples, the cells in each row are not identical to every other cell in the same row or in other rows.

An inflow apex of each hexagonal cell 30 may include an aperture 26 formed therein, which may accept sutures or similar features which may help couple other elements, such as an inner cuff 60, outer cuff 80, and/or prosthetic leaflets 90, to the frame 20. However, in some examples, one or more or all of the apertures 26 may be omitted.

Still referring to FIG. 2, the outflow section 24 of the frame 20 may include larger cells 34 that have generally asymmetric shapes. For example, the lower or inflow part of the larger cells 34 may be defined by the two upper struts 29 of a cell 32, and one upper strut 29 of each of the two adjacent cells 32. In other words, the lower end of each larger cell 34 may be formed by a group of four consecutive upper struts 29 of three circumferentially adjacent cells 32. The tops of the larger cells 34 may each be defined by two linking struts 35a, 35b. The first linking strut 35a may couple to a top or outflow apex of a cell 32 and extend upwards at an angle toward a commissure attachment feature (“CAF”) 40. The second linking strut 35b may extend from an end of the first linking strut 35a back downwardly at an angle and connect directly to the CAF 40. To the extent that the larger cells 34 include sides, a first side is defined by a portion of the CAF 40, and a second side is defined by the connection between first linking strut 35a and the corresponding upper strut 29 of the cell 32 attached to the first linking strut 35a.

The CAF 40 may generally serve as an attachment site for leaflet commissures (e.g. where two prosthetic leaflets 90 join each other) to be coupled to the frame 20. In the illustrated example, the CAF 40 is generally rectangular and has a longer axial length than circumferential width. The CAF 40 may define an interior open rectangular space. The struts that form CAF 40 may be generally smooth on the surface defining the open rectangular space, but some or all of the struts may have one or more suture notches on the opposite surfaces. For example, in the illustrated example, CAF 40 includes two side struts (on the longer side of the rectangle) and one top (or outflow) strut that all include alternating projections and notches on their exterior facing surfaces. These projections and notches may help maintain the position of one or more sutures that wrap around these struts. These sutures may directly couple the prosthetic leaflets 90 to the frame 20, and/or may directly couple an intermediate sheet of material (e.g., fabric or tissue) to the CAF 40, with the prosthetic leaflets 90 being directly coupled to that intermediate sheet of material. In some embodiments, tabs or ends of the prosthetic leaflets 90 may be pulled through the opening of the CAF 40, but in other embodiments the prosthetic leaflets 90 may remain mostly or entirely within the inner diameter of the frame 20. It should be understood that balloon-expandable frames are typically formed of metal or metal alloys that are very stiff, particularly in comparison to self-expanding frames. At least in part because of this stiffness, although the prosthetic leaflets 90 may be sutured or otherwise directly coupled to the frame at the CAFs 40, it may be preferable that most or all of the remaining portions of the prosthetic leaflets 90 are not attached directly to the frame 20, but are rather attached directly to an inner skirt 60, which in turn is directly connected to the frame 20. Further, it should be understood that other shapes and configurations of CAFs 40 may be appropriate. For example, various other suitable configurations of frames and CAFs are described in greater detail in U.S. Provisional Patent Application No. 63/579,378, filed Aug. 29, 2023 and titled “TAVI Deployment Accuracy-Stent Frame Improvements,” the disclosure of which is hereby incorporated by reference herein.

With the example described above, frame 20 includes two rows of hexagon-shaped cells 30, 32, and a single row of larger cells 34. In a three-leaflet embodiment of a prosthetic heart valve that incorporates frame 20, each row of hexagon-shaped cells 30, 32 includes twelve cells, while the row of larger cells includes six larger cells 34. As should be understood, the area defined by each individual cell 30, 32 is significantly smaller than the area defined by each larger cell 34 when the frame 20 is expanded. There is also significantly more structure (e.g., struts) that create each row of individual cells 30, 32 than structure that creates the row of larger cells 34.

One consequence of the above-described configuration is that the inflow section 22 has a higher cell density than the outflow section 24. In other words, the total numbers of cells, as well as the number of cells per row of cells, is greater in the inflow section 22 compared to the outflow section 24. The configuration of frame 20 described above may also result in the inflow section 22 being generally stiffer than the outflow section 24 and/or more radial force being required to expand the inflow section 22 compared to the outflow section 24, despite the fact that the frame 20 may be formed of the same metal or metal alloy throughout. This increased rigidity or stiffness of the inflow section 22 may assist with anchoring the frame 20, for example after balloon expansion, into the native heart valve annulus. The larger cells 34 in the outflow section 24 may assist in providing clearance to the coronary arteries after implantation of the prosthetic heart valve 10. For example, after implantation, one or more coronary ostia may be positioned above the frame 20, for example above the valley where two adjacent larger cells 34 meet (about halfway (+/−15 degrees) between a pair of circumferentially adjacent CAFs 40). Otherwise, one or more coronary ostia may be positioned in alignment with part of the large interior area of a larger cell 34 after implantation. Either way, blood flow to the coronary arteries is not obstructed, and a further procedure that utilizes the coronary arteries (e.g. coronary artery stenting) will not be obstructed by material of the frame 20. Still further, the lower rigidity of the frame 20 in the outflow section 24 may cause the outflow section 24 to preferentially foreshorten during expansion, with the inflow section 22 undergoing a relatively smaller amount of axial foreshortening. This may be desirable because, as the prosthetic heart valve 10 expands, the position of the inflow end of the frame 20 may remain substantially constant relative to the native valve annulus, which may make the deployment of the prosthetic heart valve 10 more precise. This may be, for example, because the inflow end of the frame 20 is typically used to gauge proper alignment with the native valve annulus prior to deployment, so axial movement of the inflow end of the frame 20 relative to the native valve annulus during deployment may make precise placement more difficult.

Referring back to FIG. 1, the prosthetic heart valve 10 may include an inner skirt 60 mounted to the interior surface of frame 20. The inner skirt 60 may be formed of tissue, such as pericardium, although other types of tissue may be suitable. In the illustrated example, the inner skirt 60 is formed of a woven synthetic fabric, such as polyethylene terephthalate (“PET”) or polytetrafluoroethylene (“PTFE”), although other fabrics may be suitable, including fabrics other than woven fabrics. In some examples, the inner skirt 60 has straight or zig-zag shaped inflow and outflow ends that generally follow the contours of the cells 30, 32 of the inflow section 22 of frame 20. Preferably, inner skirt 60 is sutured to the frame 20 along the struts that form cells 30, 32. If apertures 26 are included, inner skirt 60 may also be coupled to frame 20 via sutures passing through apertures 26. Preferably, the inner skirt 60 does not cover (or does not cover significant portions of) the larger cells 34. The inner skirt 60 may be coupled to the frame 20 via mechanisms other than sutures, including for example ultrasonic welding or adhesives. Further, the inner skirt 60 may have shapes other than that shown, and need not have a zig-zag inflow or outflow end, and need not cover every cell in the inflow section 22. In fact, in some examples, the inner skirt 60 may be omitted entirely, with the outer skirt 80 (described in greater detail below) being the only skirt used with prosthetic heart valve 10. If the inner skirt 60 is provided, it may assist with sealing the prosthetic heart valve 10 within the heart, as well as serving as a mounting structure for the prosthetic leaflets 90 (described in greater detail below) within the frame 20.

Still referring to FIG. 1, the prosthetic heart valve 10 may include an outer skirt 60 mounted to the exterior surface of frame 20. The outer skirt 80 may be formed of tissue, such as pericardium, although other types of tissue may be suitable. In the illustrated example, the outer skirt 80 is formed of a woven synthetic fabric, such as PET or PTFE, although other fabrics may be suitable, including fabrics other than woven fabrics. In some examples, the outer skirt 80 has straight or zig-zag inflow end. Preferably, outer skirt 80 is sutured to the frame 20 and/or inner skirt 60 along the inflow edge of the outer skirt 80. If apertures 26 are included, outer skirt 80 may also be coupled to frame 20 via sutures passing through apertures 26. The outer skirt 80 may include a plurality of folds or pleats, such a circumferentially extending folds or pleats. The folds or pleats may be formed in the outer skirt 80 via heat setting, for example by placing the outer skirt 80 within a mold that forces the outer skirt 80 to form folds of pleats, and the outer skirt 80 may be treated with heat so that the outer skirt 80 tends to maintain folds or pleats in the absence of applied forces. The outflow edge of outer skirt 80 may be coupled to the frame 20 at selected, spaced apart locations around the circumference of the frame 20. In some embodiments, the outflow edge of outer skirt 80 may be connected to the inner skirt 60 along a substantially continuous suture line. Some or all of the outer skirt 80 between its inflow and outflow edges may remain not directly couples to the frame 20 or inner skirt 60. Preferably, the outer skirt 80 does not cover (or does not cover significant portions of) the larger cells 34. In use, the outer skirt 80 may directly contact the interior surface of the native heart valve annulus to assist with sealing, including sealing against PV leak. If folds or pleats are included with the outer skirt 80, the additional material of the folds or pleats may help further mitigate PV leak. However, it should be understood that the folds or pleats may be omitted from outer skirt 80, and the outer skirt 80 may have shapes other than that shown. In fact, in some examples, the outer skirt 80 may be omitted entirely, with the inner skirt 60 being the only skirt used with prosthetic heart valve 10. If the inner skirt 60 is omitted, the prosthetic leaflets 90 may be attached directly to the frame 20 and/or directly to the outer skirt 80.

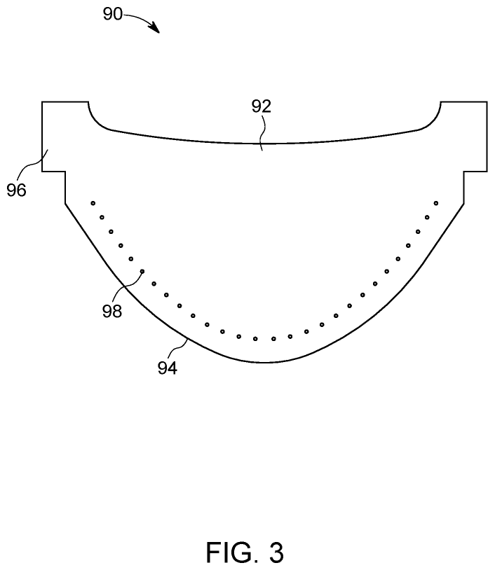

FIG. 3 is a front view of a prosthetic leaflet 90, as if laid flat on a table. In the illustrated example of prosthetic heart valve 10, a total of three prosthetic leaflets 90 are provided, although it should be understood that more or fewer than three prosthetic leaflets may be provided in other example of prosthetic heart valves. The prosthetic leaflet 90 may be formed of a synthetic material, such a polymer sheet or woven fabric, or a biological material, such a bovine or porcine pericardial tissue. However, other materials may be suitable. In on example, the prosthetic leaflet 90 is formed to have a concave free edge 92 configured to coapt with the free edges of the other leaflets to help provide the one-way valve functionality. The prosthetic leaflet 90 may include an attached edge 94 which is attached (e.g., via suturing) to other structures of the prosthetic heart valve 10. For example, the attached edge 94 may be coupled directly to the inner skirt 60, directly to the frame 20, and/or directly to the outer skirt 80. It may be preferable that the attached edge 94 is coupled directly only to the inner skirt 60, which may help reduce stresses on the prosthetic leaflet 90 compared to if the attached edge 94 were coupled directly to the frame 20. In some embodiments, a plurality of holes 98 may be formed along the attached edge 94 (or a spaced distance therefrom), for example via lasers. If included, the holes 98 may be used to receive sutures therethrough, which may make it easier to couple the prosthetic leaflet 90 to the inner skirt 60 during manufacturing. For example, the holes 98 may serve as guides if suturing is performed manually, and if the positions of the holes 98 are controlled via the use of layers, the holes 98 may be consistently placed among different prosthetic leaflets 90 to reduce variability between different prosthetic leaflets 90. Laflet tabs 96 may be provided at the junctions between the free edge 92 and the attached edge 94. Each leaflet tab 96 may be joined to a leaflet tab of an adjacent prosthetic leaflet to form prosthetic leaflet commissures, which may be coupled to the frame 20 via CAFs 40.

The prosthetic heart valve 10 may be delivered via any suitable transvascular route, for example transapically or transfemorally. Generally, transapical delivery utilizes a relatively stiff catheter that pierces the apex of the left ventricle through the chest of the patient, inflicting a relatively higher degree of trauma compared to transfemoral delivery. In a transfemoral delivery, a delivery device housing or supporting the valve is inserted through the femoral artery and advanced against the flow of blood to the left ventricle. In either method of delivery, the valve may first be collapsed over an expandable balloon while the expandable balloon is deflated. The balloon may be coupled to or disposed within a delivery system, which may transport the valve through the body and heart to reach the aortic valve, with the valve being disposed over the balloon (and, in some circumstances, under an overlying sheath). Upon arrival at or adjacent to the aortic valve, a surgeon or operator of the delivery system may align the prosthetic valve as desired within the native valve annulus while the prosthetic valve is collapsed over the balloon. When the desired alignment is achieved, the overlying sheath, if included, may be withdrawn (or advanced) to uncover the prosthetic valve, and the balloon may then be expanded causing the prosthetic valve to expand in the radial direction, with at least a portion of the prosthetic valve foreshortening in the axial direction.

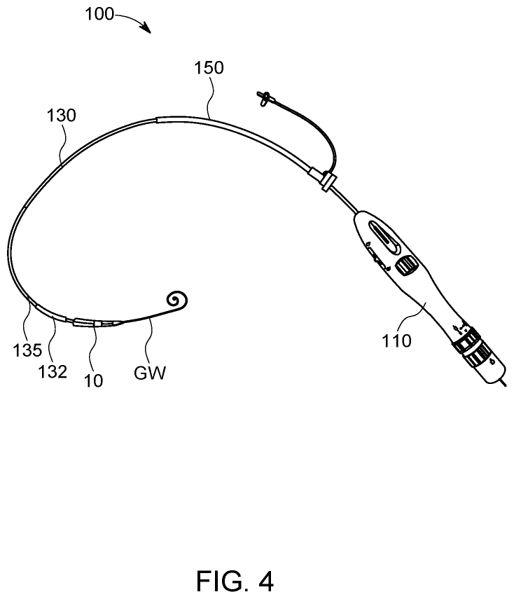

FIG. 4 illustrates one example of a delivery system 100, with the prosthetic heart valve 10 crimped over a balloon on a distal end of the delivery system 100. Although delivery system 100 and various components thereof are described below, it should be understood that delivery system 100 is merely one example of a balloon catheter that may be appropriate for use in delivering and deploying prosthetic heart valve 10.

In some examples, delivery system 100 includes a handle 110 and a delivery catheter 130 extending distally from the handle 110. An introducer 150 may be provided with the delivery system 100. Introducer 150 may be an integrated or captive introducer, although in other embodiments introducer 150 may be a non-integrated or non-captive introducer. In some examples, the introducer 150 may be an expandable introducer, including for example an introducer that expands locally as a large diameter components passes through the introducer, with the introducer returning to a smaller diameter once the large diameter components passes through the introducer. In other examples, the introducer 150 is a non-expandable introducer.

A guidewire GW may be provided that extends through the interior of all components of the delivery system 100, from the proximal end of the handle 110 through the atraumatic distal tip 138 of the delivery catheter 130. The guidewire GW may be introduced into the patient to the desired location, and the delivery system 100 may be introduced over the guidewire GW to help guide the delivery catheter 130 through the patient's vasculature over the guidewire GW.

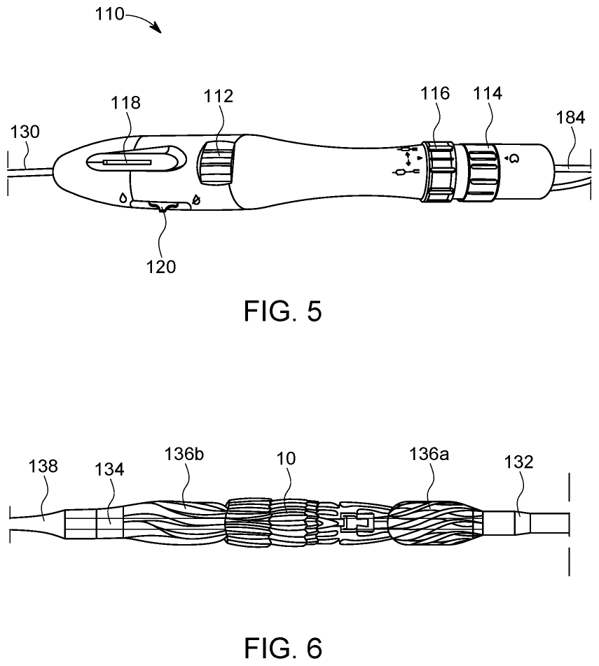

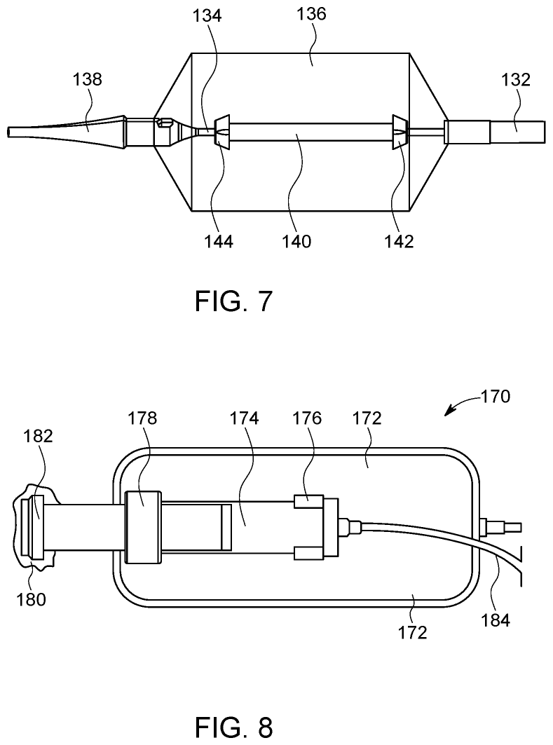

In some examples, the delivery catheter 130 is steerable. For example, one or more steering wires may extend through a wall of the delivery catheter 130, with one end of the steering wire coupled to a steering ring coupled to the delivery catheter 130, and another end of the steering wire operable coupled to a steering actuator on the handle 110. In such examples, as the steering actuator is actuated, the steering wire is tensioned or relaxed to cause deflection or straightening of the delivery catheter 130 to assist with steering the delivery catheter 130 to the desired position within the patient. For example, FIG. 5 is an enlarged view of the handle 110. Handle 110 may include a steering knob 112 that, upon rotation, tensions or relaxes the steering wires to deflect the distal end of the delivery catheter 130. Handle 110 may include a slot 118 with an indicator extending therethrough, the indicator moving along the slot 118 as the delivery catheter 130 deflects (e.g., the indicator moves proximally as deflection increases). If included, the indicator and slot 118 may provide the user an easy reference of how much the delivery catheter 130 is deflected at any given point. However, it should be understood that the steering functionality may be omitted in some examples, and in other examples steering actuators other than knobs may be utilized. Further, in some examples, including those shown in FIGS. 6-7, the delivery catheter 130 includes an outer catheter 132, and an inner catheter 134. The inner catheter 134 may also be referred to as a guidewire catheter. The steering functionality may be provided in either the outer catheter 132, or the inner catheter 134, or in both catheters. However, in some examples, a separate steering catheter 135 may be provided. For example, as shown in FIG. 4, the steering catheter 135 may be positioned outside of the outer catheter 132 and may terminate just proximal to the balloon 136. With this configuration, deflection of the steering catheter 135 will also cause deflection of the outer catheter 132 and the inner catheter 134 which are both nested within the steering catheter 135.

Still referring to FIGS. 4-5, the delivery system 100 may include additional functionality to assist with positioning the prosthetic heart valve 10. For example, in the illustrated example, handle 110 includes a commissure alignment actuator 114, which may be positioned near a proximal end of the handle or at any other desired location. In the illustrated example, the commissure alignment actuator 114 is in the form of a rotatable knob, although other forms may be suitable. The commissure alignment knob 114 may be rotationally coupled to a portion of the delivery catheter 130 supporting the prosthetic heart valve 10. For example, the commissure alignment actuator 114 may be rotationally coupled to an inner catheter 134 which supports the prosthetic heart valve 10 in the crimped condition. With this configuration, rotating the commissure alignment knob 114 may cause the inner catheter 134 to rotate about its longitudinal axis, and thus cause the prosthetic heart valve 10 to rotate about its longitudinal axis. If a commissure alignment actuator 114 is included, it may be used to help ensure that, upon deployment of the prosthetic heart valve 10 into the native valve annulus, the commissures of the prosthetic heart valve are in rotational alignment with respective ones of the native valve commissures (e.g. within +/−2.5 degrees of rotational alignment, within +/−5 degrees of rotational alignment, within +/−10 degrees of rotational alignment, within +/−15 degrees of rotational alignment, etc.). Although commissure alignment actuator 114 is shown in this example as a knob positioned at or near a proximal end of the handle 110, it should be understood that the actuator 114 may take forms other than a knob, may be positioned at other suitable locations, and may be omitted entirely if desired.

Still referring to FIGS. 4-5, the delivery system 100 may include even further functionality to assist with positioning the prosthetic heart valve 10. For example, in the illustrated example, handle 110 includes an axial alignment actuator 116, which may be positioned near a proximal end of the handle, including distal to the commissure alignment actuator 114, or at any other desired location. In the illustrated example, the axial alignment actuator 116 is in the form of a rotatable knob, although other forms may be suitable. The axial alignment knob 116 may be operably coupled to a portion of the delivery catheter 130 supporting the prosthetic heart valve 10. For example, the axial alignment actuator 116 may include internal threads that engage external threads of a carriage that is coupled to the inner catheter 134 which supports the prosthetic heart valve 10 in the crimped condition. In such an example, the carriage may be rotatably fixed to the handle 110. With this configuration, rotating the axial alignment knob 116 may cause the carriage to advance distally or retract proximally as the inner threads of the axial alignment knob 116 mesh with the external threads of the carriage, but the carriage is prevented from rotating. As the carriage advances distally or retracts proximally, the inner catheter 134 may correspondingly advance distally or retract proximally, and thus cause the prosthetic heart valve 10 to advanced distally or retract proximally. It should be understood that, if axial alignment actuator 116 is included, it may have a small total range of motion. In other words, the rough or coarse axial alignment between the prosthetic heart valve 10 and native valve annulus may be achieved by physically advancing the entire delivery catheter 130 by pushing it through the vasculature while holding the handle 110. However, for fine and more controlled adjustment of the axial position of the prosthetic heart valve 10 relative to the native valve annulus, which may be performed just prior to or during deployment of the prosthetic heart valve 10, the axial alignment knob 116 may be used. If an axial alignment actuator 116 is included, it may be used to help ensure that, upon deployment of the prosthetic heart valve 10 into the native valve annulus, the inflow end of the of the prosthetic heart valve is in axial alignment with the inflow aspect of the native valve annulus (e.g. within +/−0.5 mm of axial alignment, within +/−1.0 mm of axial alignment, within +/−1.5 mm of axial alignment, within +/−2.0 mm of axial alignment, etc.). Although axial alignment actuator 116 is shown in this example as a knob positioned at or near a proximal end of the handle 110, it should be understood that the actuator 116 may take forms other than a knob, may be positioned at other suitable locations, and may be omitted entirely if desired.

In addition to steering and positioning actuators, delivery system 100 may include a balloon actuator 120. In the illustrated example, balloon actuator 120 is positioned on the handle 110 near a distal end thereof, and is provided in the form of a switch. Balloon actuator 120 may be actuated to cause inflation or deflation of a balloon 136 that is part of the delivery system 100. For example, referring briefly to FIGS. 6-7, the delivery system 100 may include a balloon 136 that overlies a distal end of inner catheter 134 and which receives the prosthetic heart valve 10 in a crimped condition thereon. In the example illustrated in FIG. 6, the balloon 136 includes a proximal pillowed portion 136a, a distal pillowed portion 136b, and a central portion over which the prosthetic heart valve 10 is crimped. The proximal pillow 136a and the distal pillow 136b may form shoulders on each side of the prosthetic heart valve 10, which may help ensure the prosthetic heart valve 10 does not move axially relative to the balloon 136 and/or inner catheter 134 during delivery. The shoulder formed by the distal pillow 136 may also help protect the inflow edge of the prosthetic heart valve 10 from contact with the anatomy during delivery. For example, during a transfemoral delivery, as the distal end of the delivery catheter 130 traverse the sharp bends of the aortic arch (or during initial introduction into the patient), there is a relatively high likelihood the inflow end of the prosthetic heart valve 10 (which is the leading edge during transfemoral delivery) will contact a vessel wall (or a components of an introduction system) causing dislodgment of the prosthetic heart valve 10 relative to the balloon 136. The distal pillow 136 may tend to have an equal or larger outer diameter than the inflow end of the prosthetic heart valve 10 (when the prosthetic heart valve 10 is crimped and the balloon 136 is deflated), which may help ensure the inflow edge of the prosthetic heart valve 10 does not inadvertently contact another structure during delivery. In some examples, the pillowed portions 136a, 136b may be formed via heat setting. Additional related features for use in similar balloon catheter delivery systems are described in greater detail in U.S. Provisional Patent Application No. 63/382,812, filed Nov. 8, 2022 and titled “Prosthetic Heart Valve Delivery and Trackability,” the disclosure of which is hereby incorporated by reference herein.

In order to deploy the prosthetic heart valve 10, the balloon 136 is inflated, for example by actuating the balloon actuator 120 to force fluid (such as saline, although other fluids, including liquids or gases, could be used) into the balloon 136 to cause it to expand, causing the prosthetic heart valve 10 to expand in the process. For example, the balloon actuator 120 may be pressed forward or distally to cause fluid to travel through an inflation lumen within delivery catheter 130 to inflate the balloon 136. FIG. 7 illustrates an example of the balloon 136 after being inflated, with the prosthetic heart valve 10 omitted from the figure for clarity. In the illustrated example, the balloon 136 may be formed to have a distal end that is fixed to a portion of an atraumatic distal tip 138. The distal tip 138 may be tapered to help the delivery catheter 130 move through the patient's vasculature more smoothly. A proximal end of the balloon 136 may be fixed to a distal end of outer catheter 132. The inflation lumen may be the space between the outer catheter 132 and the inner catheter 134, or in other embodiments may be provided in a wall of the inner catheter 134, or in any other location that fluidly connects the interior of the balloon 136 to a fluid source outside of the patient that is operable coupled to the delivery system 100.

Referring to FIG. 7, in some examples, a mounting shaft 140 may be provided on the inner catheter 134. A proximal stop 142 and/or a distal stop 144 may be provided, for example at opposite ends of the mounting shaft 140. If the mounting shaft 140 is included, it may provide a location on which the prosthetic heart valve 10 may be crimped. If the proximal stop 142 and/or distal stop 144 is provided, they may provide physical barriers to the prosthetic heart valve 10 moving axially relative to the balloon 136. In one example, the proximal stop 142 may taper from a larger distal diameter to a smaller proximal diameter, and the distal stop may taper from a larger proximal diameter to a smaller distal diameter. The spacing between the proximal stop 142 and the distal stop 144, if both are included, may be slightly larger than the length of the prosthetic heart valve 10 when it is crimped over mounting shaft 140. However, it should be understood that one or both of the stops 142, 144 may be omitted, and the mounting shaft 140 may also be omitted. If the mounting shaft 140 is included, it is preferably axially and rotationally fixed to the inner catheter 134 so that movement of the inner catheter 134 causes corresponding movement of the mounting member 140, and thus the prosthetic heart valve 10 when mounted thereon.

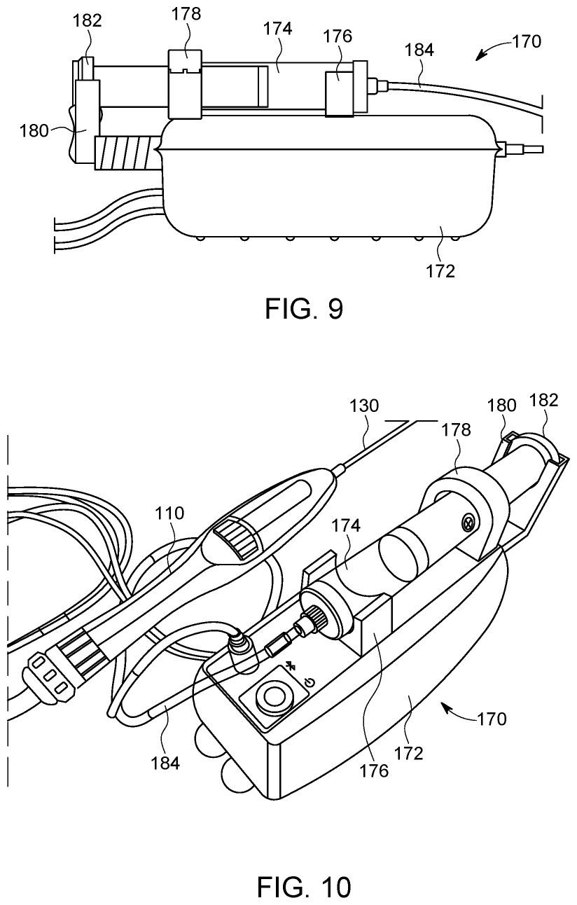

Before describing the use of balloon actuator 120 in more detail, it should be understood that in some embodiments, the balloon actuator 120 may be omitted and instead a manual device, such as a manual syringe, may be provided along with delivery system 100 in order to manually push fluid into balloon 136 during deployment of the prosthetic heart valve 10. As used herein, the phrase “fluid reservoir” and “syringe” may be used interchangeably. However, in the illustrated example of delivery system 100, the balloon actuator 120 provides for a motorized and/or automated (or semi-automated) balloon inflation functionality. For example, FIG. 8 and FIG. 9 illustrate an example of a balloon inflation system 170. Balloon inflation system 170 may include a housing 172 that houses one or more components, which may include a motor, one or more batteries, electronics for control and/or communication with other components, etc. Housing 172 may include one or more fixed cradles to receive a syringe 174. In the illustrated embodiment, a distal cradle 176 is provide with an open “C”- or “U”-shaped configuration so that the distal end of the syringe 174 may be snapped into or out of the distal cradle 176. A proximal cradle 178 may also be provided, which may have a “C”- or “U”-shaped bottom portion hingedly connected to a “C”- or “U”-shaped top portion. This configuration may allow for the proximal end of the outer body of the syringe 174 to be snapped into the bottom portion of proximal cradle 178, and the top portion of proximal cradle 178 may be closed and connected to the bottom portion to fully circumscribe the outer body of the syringe 174 to lock the syringe 174 to the housing 172. It should be understood that more or fewer cradles, of similar or different designs, may be included with housing 172 to help secure the syringe 174 to the housing 172 in any suitable fashion.

The balloon inflation system 170 may include a moving member 180. In the illustrated embodiment, moving member 180 includes a “C”- or “U”-shaped cradle to receive a plunger handle 182 of the syringe 174 therein, the cradle being attached to a carriage that extends at least partially into the housing 172. The carriage of the moving member 180 may be generally cylindrical, and may include internal threading that mates with external threading of a screw mechanism (not shown) within the housing 172 that is operably coupled to a motor. In some embodiments, the carriage may have the general shape of a “U”-beam with the flat face oriented toward the top. The moving member 180 may be rotationally fixed to the housing 172 via any desirable mechanism, so that upon rotation of the screw mechanism by the motor, the moving member 180 advances farther into the housing 172, or retracts farther away from the housing 172, depending on the direction of rotation of the screw mechanism. While the plunger handle 182 is coupled to the moving member 180, advancement of the moving member 180 forces fluid from the syringe 174 toward the balloon 136, while retraction of the moving member 180 withdraws fluid from the balloon 136 toward the syringe 174. It should be understood that the motor, or other driving mechanism, may be located in or outside the housing 172, and any other suitable mechanism may be used to operably couple the motor or other driving mechanism to the moving member 180 to allow for axial driving of the plunger handle 182.

As shown in each of FIG. 8, FIG. 9, and FIG. 10, the distal end of syringe 174 may be coupled to tubing 184 that is in fluid communication with an inflation lumen of delivery catheter 130 that leads to the balloon 136 at or near the distal end of the delivery system 100. Tubing 184 may allow for the passage of the fluid (e.g., saline) from the syringe 174 toward the balloon 136, or for withdrawal of fluid from the balloon 136 toward the syringe 174, for example based on whether the balloon actuator 120 is pressed forward or backward.

Although not separately numbered in FIG. 8, FIG. 9, and FIG. 10, the housing 172 may include one or more cables extending from the housing, for example to allow for transmission of power (e.g., from AC mains or another component with which the cable is coupled) and/or transmission of data, information, control commands, etc. For example, one cable may couple the housing 172 to handle 110 so that controls on the handle 110 (e.g., balloon actuator 120) may be used to activate the balloon inflation system 170 in the desired fashion. Another cable may couple to a computer display or similar device to provide information regarding the inflation of the balloon 136. However, it should be understood that any transmission of data or information may be provided wirelessly instead of via a wired connection, for example via a Bluetooth or other suitable connection. Additional and related features of balloon inflation system 170, related systems, and the uses thereof are described in U.S. patent application Ser. No. 18/311,458, the disclosure of which is hereby incorporated by reference herein.

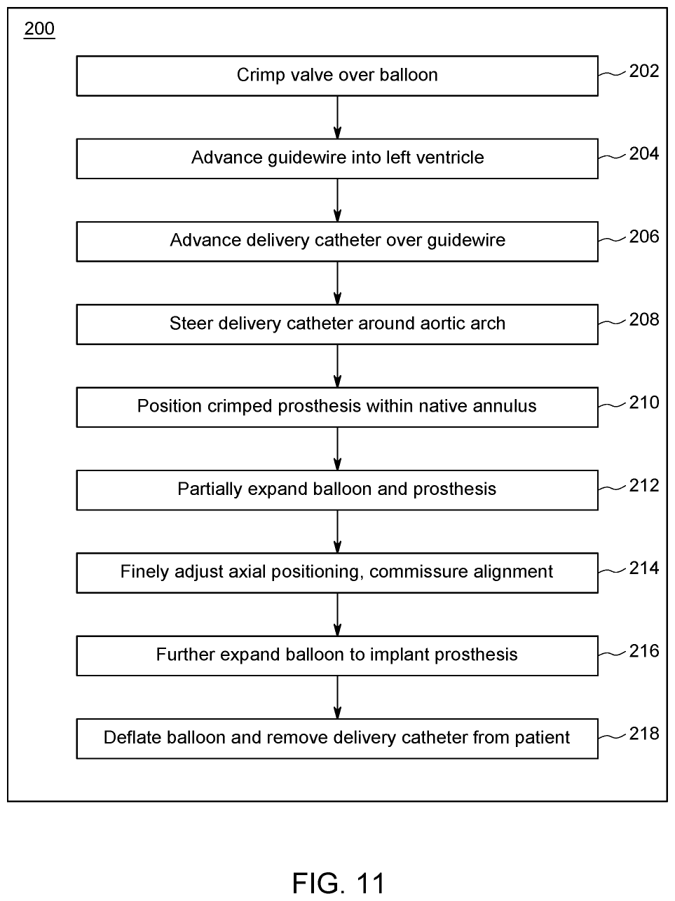

FIG. 11 is a flowchart showing exemplary steps in an implantation procedure 200 to implant the prosthetic heart valve 10 of FIG. 1 into a patient using the delivery system 100 of FIG. 4. However, it should be understood that not all of the steps shown in connection with implantation procedure 200 need to be performed, and various steps not explicitly shown and described in connection with procedure 200 may be performed as part of the implantation procedure. At the beginning of the procedure 200 in step 202, the prosthetic heart valve 10 may be collapsed over or crimped onto balloon 136, with the balloon 136 being mostly or entirely deflated after the crimping procedure. It should be understood that crimping step 202 may be performed at any time prior to the procedure, including at the beginning of the procedure, or at an earlier stage before the delivery system 100 is provided to the end user. In other words, the crimping step 202 may be performed during a manufacturing stage of the delivery system 100 and/or prosthetic heart valve 10. During an early stage of the implantation procedure 200, a guidewire GW may be advanced into the patient in step 204, for example via the femoral artery, around the aortic arch, through the native aortic valve, and into the left ventricle. The guidewire GW may be used as a rail for other devices that need to access this pathway. For example, in step 206, the atraumatic distal tip 138 may be advanced over the proximal end of the guidewire GW, and the delivery catheter 130 may be advanced over guidewire GW toward the native aortic valve. During this initial advancement of the delivery catheter 130 into the patient, the introducer 150 (if included) may be positioned distally, for example so that it covers the prosthetic heart valve 10 or so that it is positioned just proximal to the prosthetic heart valve 10. Advancement of the delivery catheter 130 and introducer 150 may continue until a proximal hub of the introducer is in contact with the patient's skin (or in contact with another device that enters the patient's femoral artery. At this point, the introducer 150 may stop moving axially relative to the patient, with the delivery catheter 130 continuing to advance relative to the introducer 150. If steering capability is provided, the delivery catheter 130 may be steered or deflected at any point to assist with achieving the desired pathway of the delivery catheter 130. As on example, in step 208, the steering knob 112 may be actuated to deflect the distal end of the delivery catheter 130 as it traverses the sharp bends of the aortic arch. Advancement of the delivery catheter 130 may continue in step 210 until the prosthetic heart valve 10, while still crimped or collapsed, is positioned within the native aortic valve annulus. With the desired position achieved, the balloon 136 may be partially inflated, for example by pressing balloon actuator 120 forward, to partially expand the prosthetic heart valve 10 in step 212. In some examples, it is desirable to expand the prosthetic heart valve 10 only partially in step 212, because the position of the prosthetic heart valve 10 (including rotational and/or axial positioning) relative to the native aortic valve annulus may shift during this partial expansion. After the partial expansion of step 212, the user may examine the positioning of the prosthetic heart valve 10 relative to the native aortic valve annulus. If desired, in step 214, the axial positioning of the partially-expanded prosthetic heart valve 10 relative to the native aortic valve annulus may be finely adjusted (e.g., by actuating axial alignment actuator 116) and/or the rotational orientation of the prosthetic heart valve 10 relative to the native aortic valve may be finely adjust (e.g., by actuating commissure alignment actuator 114). When the desired axial alignment is achieve and the desired rotational alignment (e.g., rotational alignment between the prosthetic commissure and the native commissures) is achieved, the balloon 136 may be fully expanded in step 216 to fully expand the prosthetic heart valve 10 and to anchor the prosthetic heart valve 10 in the native aortic valve annulus in the desired position and orientation. After deployment is complete, the balloon 136 may be deflated in step 218, for example by pressing actuating balloon 120 backward, and the delivery catheter 130 and guidewire GW may be removed from the patient to complete the procedure. It should be understood that the nine steps shown in FIG. 11 as part of procedure 200 are merely exemplary of a single example of an implantation procedure, and steps shown may be omitted, steps not shown may be included, and steps may be provided in any order deemed appropriate by the physician and/or medical personnel. In one example, the delivery catheter 130 may be guided to the right atrium and/or right ventricle for a tricuspid valve or pulmonary valve procedure. In another example, the delivery catheter 130 may be guided to the left atrium and/or left ventricle for a mitral valve procedure.

Although various components of a prosthetic heart valve 10 and delivery system 100 are described above, it should be understood that these components are merely intended to provide better context to the systems, features, and/or methods described below. Thus, various components of the systems described above may be modified or omitted as appropriate without affecting the systems, features, and/or methods described below. For example, prosthetic heart valves other than the specific configuration shown and described in connection with FIGS. 1-3 may be used with delivery systems other than the specific configuration shown and described in connection with FIGS. 4-10 as part of an implantation procedure that uses steps other than the specific configuration shown and described in connection with FIG. 11, without affecting the inventive systems, features, and/or methods described below.

In some examples, delivery system 100 (or other prosthetic heart valve delivery systems) may be provided to the end user (e.g., a hospital, clinic, physician, etc.) with prosthetic heart valve 10 (or another balloon-expandable prosthetic heart valve) “pre-crimped” over balloon 136. As used herein, the term “pre-crimped” refers to the scenario in which the prosthetic heart valve (e.g., valve 10) is already crimped over the balloon (e.g., balloon 136) of the delivery system (e.g., delivery system 100) at the time of receipt of the system by the end user (or an individual, location or institution associated with the end user). Providing the delivery system 100 to the end user with the prosthetic heart valve 10 pre-crimped over the balloon may reduce the amount of time and/or effort required by the end user to prepare the delivery system 100 for use. In other words, the step for crimping the prosthetic heart valve 10 over the balloon 136 may be performed by the supplier of the system, instead of the user of the system which is currently the typical procedure. If a user receives the delivery system 100 with the prosthetic heart valve 10 pre-crimped on the balloon 136, the only additional preparation of the delivery system 100 needed to be performed by the end user may be flushing and/or de-airing fluid lines and/or the balloon 136 of the delivery system 100. As transcatheter prosthetic heart valve procedures increase in prevalence, it may become more important to be able to reduce the time required per procedure to allow more cases to be treated within a given time period. It is also generally important for the largest part of the delivery system 100 that needs to pass through the patient's vasculature to be relatively small. Because transcatheter prosthetic heart valve implantations are typically performed through the vasculature, as the size of the system within the vasculature gets larger, the likelihood of complications, such as causing damage to the native tissue, may increase. The location of the prosthetic heart valve where it is crimped over the delivery system is often the largest profile portion of the device that will pass through the vasculature. As used herein, the phrase “crimp profile” refers to the outer diameter or profile of the prosthetic heart valve 10 when it is crimped on the delivery system 100 (e.g., over balloon 136), in a state ready for delivery into and/or through the patient's vasculature. At least some of the disclosure provided herein may address achieving one or more of these goals.

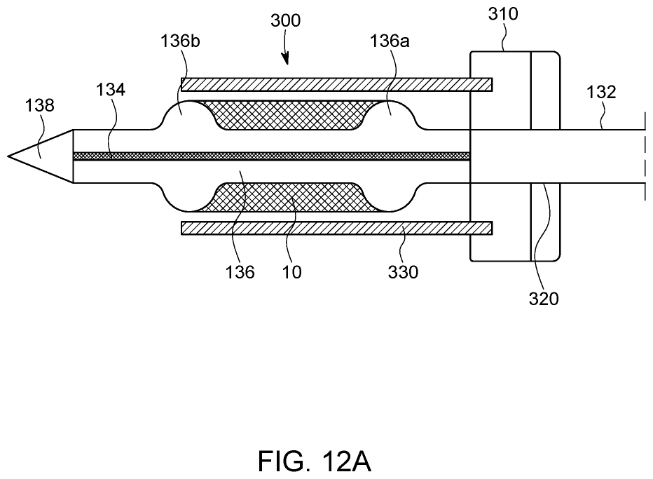

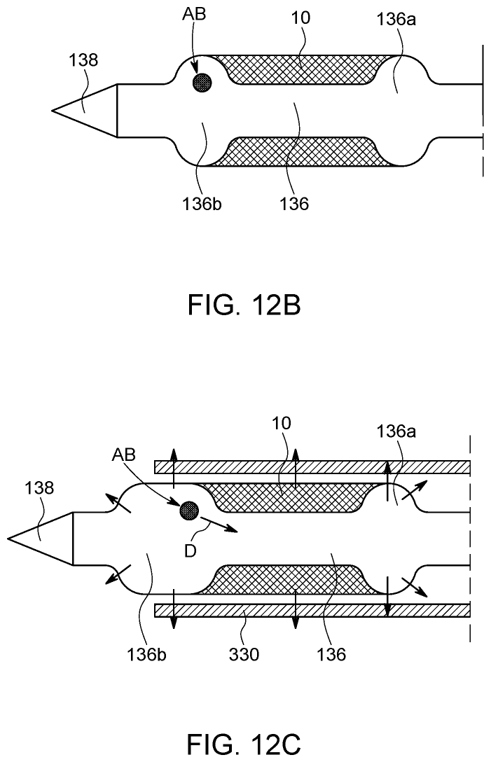

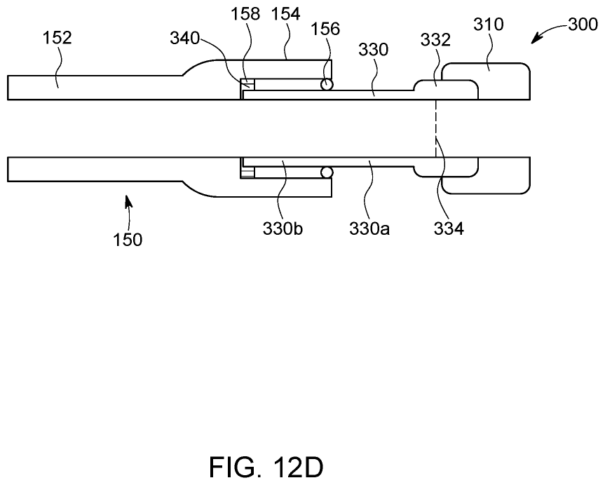

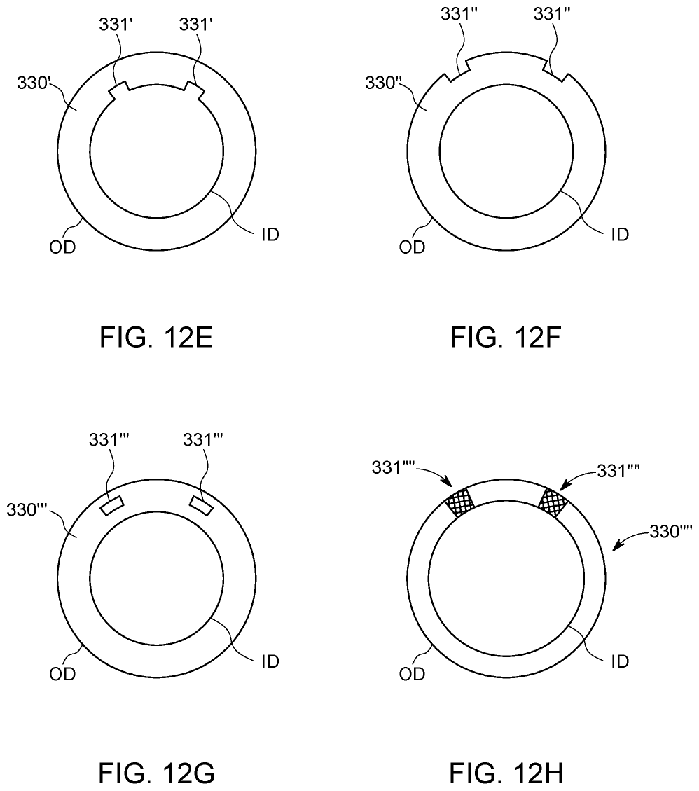

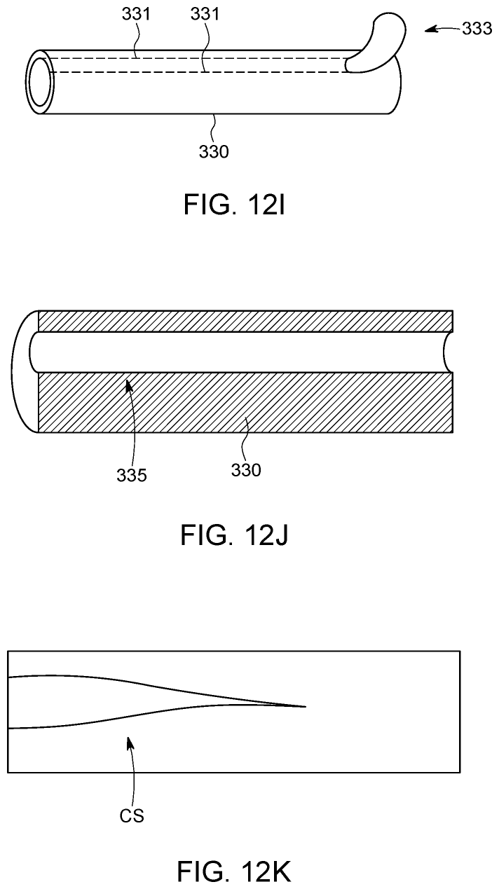

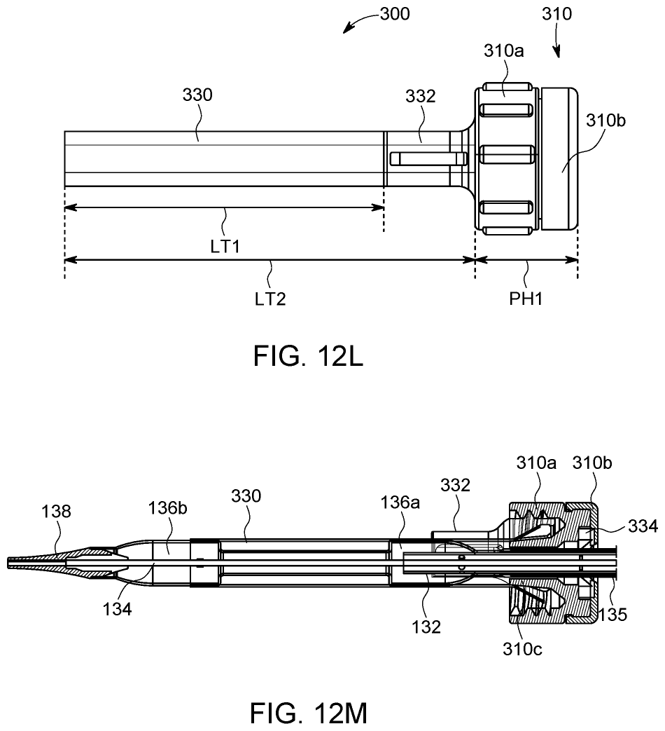

Referring now in addition to FIG. 12A, FIG. 12A is a cross-section of the distal end of delivery system 100 with an example of a loader sheath 300 assembled thereto. In the example of FIG. 12A, the prosthetic heart valve 10 is already crimped over the balloon 136, between proximal pillow 136a and distal pillow 136b. In the illustrated example, the loader sheath 300 includes a proximal cap or hub 310, which may include a lumen 320 therethrough. In some examples, the lumen 320 may be sized and shaped to receive the outer catheter 132 (and/or the steering catheter 135) therethrough. In the illustrated example, the loader sheath includes a tube 330 extending distally from the hub 310. In some examples, the tube 330 may be integral with the hub 310, and in other examples the tube 330 may be formed separately from and coupled to the hub 310. In some examples, the hub 310 may be generally cylindrical (or annular), and the tube 330 may be generally cylindrical (or annular), and in some examples the hub 310 may have a larger outer diameter than the tube 330. In some examples, the tube 330 is formed of a rigid and/or non-compliant material, including for example fluorinated ethylene propylene (“FEP”), polytetrafluoroethylene (“PTFE”), nylon, or high density polyethylene (“HDPE”). It should be understood that, unless explicitly mentioned otherwise, all examples of tubes of a loader sheath described herein may be formed of a rigid and/or non-compliant material. In some examples, the hub 310 is formed of the same material as the tube 330, although in other examples the hub 310 may be formed of a different material than the tube 330. In some examples, the tube 330 of the loader sheath 300 has an inner diameter that is about equal to (+/−2.5%), slightly smaller than (e.g., up to 5% smaller than), or slightly larger than (e.g., up to 5% larger than) the crimp profile of the prosthetic heart valve 10. In the examples in which the inner diameter of the tube 330 is smaller than the crimp profile of the prosthetic heart valve 10, the prosthetic heart valve 10, when received within the tube 330, may be maintained with a slightly smaller crimp profile than it would have had in the absence of the tube 330 being positioned over the prosthetic heart valve 10.

In one example, loader sheath 300 may be positioned over the crimped prosthetic heart valve 10, including similar to as shown in FIG. 12A, after the prosthetic heart valve 10 has been crimped over balloon 136, but prior to being shipped or otherwise delivered to the end user. For example, prosthetic heart valve 10 may be crimped over balloon 136 by the manufacturer, as opposed to by the end user immediately prior to use. With this configuration, the crimp process may be more precisely and thoroughly controlled, reducing user-to-user variability in how the prosthetic heart valve 10 is crimped over the balloon 136. However, because components of the prosthetic heart valve 10 may have some fragility, it may be important to protect the prosthetic heart valve 10 from damage during shipment or otherwise between the delivery system 100 being initially assembled and the time at which it is used in a procedure. If the loader sheath 300 is assembled with the delivery system 100 with the tube 330 covering the prosthetic heart valve 10 while the prosthetic heart valve 10 is crimped over the balloon 136, the loader sheath 300, including the tube 330, may help to protect the prosthetic heart valve 10 from damage during the time period between initial assembly (e.g., at a manufacturing or assembly facility) and its final use (e.g., in a hospital and/or cath lab). In some examples, during most or all of this time period, the delivery system 100 may be provided in a packaging, and thus the loader sheath 300 may help provide additional protection while the delivery system 100, including prosthetic heart valve 10, are in a packaged state or packaged configuration.

The loader sheath 300 may in some examples provide additional functionality, as described in greater detail below. However, it should be understood that any of the functionality of the loader sheath 300 may be exploited individually or in any combination. For example, although loader sheath 300 is described above as providing protection to the prosthetic heart valve 10 if the prosthetic heart valve 10 is pre-mounted to the delivery system 100, other possible benefits described in greater detail below may be realized even if the prosthetic heart valve 10 is not provided in a pre-crimped or pre-mounted configuration.

Now referring in addition to FIG. 12B, FIG. 12B is a highly schematic view of an example of an air bubble AB trapped within the distal pillow 136b of balloon 136, while prosthetic heart valve 10 is crimped over balloon 136, but in the absence of any loader sheath (e.g., loader sheath 300) overlying the prosthetic heart valve 10. It is typically desirable to remove any air bubbles from within the fluid line that leads to balloon 136. For example, if balloon 136 were to burst while inside the body during a procedure, and if an air bubble were released into the bloodstream, significant trauma including stroke could result. At least for this reason, in typical prosthetic heart valve implantations using an expandable balloon, attempts are made to eliminate or at least minimize the amount of ai within the fluid line that leads to the balloon. Referring to FIG. 12B, one potential challenge with removing air bubble AB from balloon 136 when prosthetic heart valve 10 is already crimped on the balloon 136 is that the air bubble AB may get “caught” within the distal pillow 136b, at least in part due to the structure of the prosthetic heart valve 10 being positioned adjacent the distal pillow 136b. Another potential challenge with removing air bubble AB from balloon 136 when prosthetic heart valve 10 is already crimped on balloon 136 is that, if fluid (e.g., saline) is being alternatively pushed into and removed from balloon 136 in an effort to remove the air bubble AB, there is a risk of unintentionally inflating the balloon 136 and forcing the prosthetic heart valve 10 to expand. If the prosthetic heart valve 10 expands during de-airing, after having being previously crimped onto the balloon 136, the crimping process may need to be repeated, which may be time consuming at best, and not practically possible at the worst.

Now referring in addition to FIG. 12C, the use of loader sheath 300 may in some examples help mitigate both potential issues described in connection with FIG. 12B. It should be understood that the examples of potential benefits described in connection with FIG. 12C may be achieved with loader sheath 300 anytime that de-airing is being performed while the prosthetic heart valve 10 is crimped over the balloon 136, whether or not the delivery system 100 is provided to the user with the prosthetic heart valve 10 in a pre-crimped configuration. In examples in which the tube 330 of the loader sheath 300 is positioned to overlie the prosthetic heart valve 10 crimped over balloon 136 during de-airing, including when the tube 330 is formed of a rigid material, the balloon 136 may be pressurized without risk that the balloon 136 will prematurely inflate and prematurely expand the prosthetic heart valve 10. For example, a syringe 174 (or other fluid reservoir) in fluid communication with the balloon 136 may be pressurized to pressurize the balloon 136. In the example of FIG. 12C, the pressurization of balloon 136 is represented by outwardly pointing arrows, with the tube 330 of loader sheath 300 preventing the balloon 136 from inflating and preventing the prosthetic heart valve 10 from expanding. In some examples, the pressurization of balloon 136 may cause the air bubble AB to compress (e.g., decrease in size), making it easier for the air bubble AB to pass proximally through the balloon 136, as represented by the directional arrow D, past the leading edge of the prosthetic heart valve 10 and through the prosthetic heart valve 10, and eventually removed from the fluid line.

Referring now in addition to FIG. 12D, FIG. 12D is a cross-section of an example of loader sheath 300 coupled to an example of the introducer 150 of the delivery system of FIG. 4. In the illustrated example, a proximal end of introducer 150 is shown, which may be an expandable introducer in some examples. In some examples, the introducer 150 may include a sheath 152, only a portion of which is visible in FIG. 12D. An introducer hub 154 in some examples may be positioned at or near a proximal end of the introducer sheath 152. In the illustrated example, the introducer hub 154 may have a larger inner diameter and/or a larger outer diameter compared to introducer sheath 152. In some examples, a hemostasis valve 156 is positioned within the introducer hub 154, although in other examples, a hemostasis valve (if included) may instead be positioned within the introducer sheath 152. Hemostasis valve 156 may help to prevent blood from leaking out of the introducer 150 when it is inserted into the vasculature, particularly when there are no components (or only relatively small-diameter components) passing through the interior of the introducer 150. In some examples, the hemostasis valve 156 may be a duckbill valve, a cross-slit valve, or an adjustable valve such as a mechanically adjustable valve or a valve which may have its size adjusted by filling a bladder that at least partially forms the valve.

In some examples, when using delivery system 100 to deliver prosthetic heart valve 10, there are no outer sheaths covering the prosthetic heart valve 10 as the prosthetic heart valve 10 is passed through the patient's vasculature. However, in such examples, it may become problematic if the prosthetic heart valve 10 is passed into the introducer 150, and particularly through the hemostasis valve 156, without any protective covering. For example, if prosthetic heart valve 10 were advanced through hemostasis valve 156 and into introducer 150 while the prosthetic heart valve 10 was crimped over balloon 136 without a protective sheath, contact between the prosthetic heart valve 10 and structure of the hemostasis valve 156 could dislodge the prosthetic heart valve 10 from its position on the balloon 136. However, if loader sheath 300 overlies the prosthetic heart valve 10, for example as shown in FIG. 12A, the tube 330 of loader sheath 300 may contact the hemostasis valve 156 while protecting the prosthetic heart valve 10 from being contacted (and potentially dislodged and/or damaged) by the hemostasis valve 156.

In some examples, including the example shown in FIG. 12D, the outer tube 330 of loader sheath 300 may have an outer diameter that is about equal to or smaller than the inner diameter of the introducer hub 154 so that the loader sheath 300 may at least partially telescope into the introducer 150. In the illustrated example, the inner diameter of the tube 330 is about equal to the inner diameter of the introducer sheath 152. With this example configuration, the prosthetic heart valve 10 and overlying loader sheath 300 may be passed through the hemostasis valve 156 together, until the distal end of the tube 330 contacts the proximal end of the introducer sheath 152 (or otherwise contacts a reduced-diameter section of the introducer hub 154), at which point the user may continue advancing the delivery catheter 130 (including the prosthetic heart valve 10) through the introducer 150 and into the patient while the loader sheath 300 remains substantially stationary relative to the introducer 150. In the illustrated example, when the loader sheath 300 is maximally inserted into the introducer 150 (as shown in the example of FIG. 12D), a distal length 330b of the tube 330 may be telescoped or otherwise contained radially within the introducer hub 154, while a proximal length 330a of the tube 330 may protrude beyond the proximal end of the introducer hub 154. Because the handle 110 of the delivery system 100 is in some examples too large to pass into or through the loader sheath 300, as the length of the protruding proximal length 330a of the tube 330 increases, the working length of the delivery catheter 130 may decrease. In other words, if 6 inches (about 15.24 cm) of structure of the loader sheath 300 protrudes proximally of the introducer hub 154 (which length may include both proximal length 330a and proximal hub 310) when the configuration of FIG. 12D is achieved, the total available working length of delivery catheter 130 may be reduced by about 6 inches (about 15.24 cm) compared to the scenario in which the loader sheath 300 is omitted. However, in examples in which the loader sheath 300 is configured to at least partially telescope into the introducer 150 (e.g., into the introducer hub 154), some of the working length of the delivery sheath 130 that might otherwise be lost may be regained. In some examples, when the loader sheath 300 is inserted into the introducer 150 until the distal end of the loader sheath 300 abuts a proximal surface of the introducer 150 (e.g., the proximal end of the introducer sheath 152 or a reduced-diameter section of the introducer hub 154), and thus the loader sheath 300 is at its maximum available insertion distance into the introducer 150, the total length of the distal tube 330b positioned within the introducer 150 is about equal to the total length of the proximal tube 330a that still protrudes beyond the introducer 150. In other words, in this example, when the configuration of FIG. 12D is achieved, about half (+/−5%) of the length of the tube 330 is telescoped within the introducer 150. However, in other embodiments, when the configuration of FIG. 12D is achieved, up to about 95% to about 100% (or about 75% (+/−5%), or about 25% (+/−5%) of the length of the tube 330 is telescoped within the introducer 150. In some examples, it may be desirable to maximize the length of the tube 330 that telescopes into the introducer 150 to minimize the reduction in working length of the delivery sheath 130.

In some examples, the loader sheath 300 includes a locking member or coupling feature, such as coupler 340. In some examples, an interior of the introducer 150, such as the interior of the introducer hub 154 and/or the proximal end of the introducer sheath 152, may include a corresponding locking member or coupling feature, such as coupler 158. If couplers 340 and 158 are provided, in some examples they may be corresponding threads that thread together, protrusions and detents, latches, hooks, or any other suitable coupling mechanism. If couplers 340 and/or 158 are provided, once the distal end of the loader sheath 300 is mostly or fully inserted into the introducer hub 154, couplers 340 may engage with couplers 158 to at least temporarily lock the position of the loader sheath 300 relative to the introducer 150. With this type of example configuration, once the loader sheath 300 is at least temporarily locked to the introducer 150, the loader sheath 300 and introducer 300 may effectively act as a single unit, which may simplify the overall procedure, for example because the loader sheath 300 may not need any further manipulation (e.g., removal) and/or because a user's single handle can manipulate the introducer sheath 150 and the loader sheath 300 simultaneously.

Still referring to FIG. 12D, in some examples, a proximal end of tube 330 may terminate in a cap 332, which may or may not have an outer diameter that is larger than the outer diameter of the tube 330. In some examples, a hemostasis valve 334 is positioned within the cap 332 (although in other examples, hemostasis valve 334 may be positioned at other locations within tube 330). As with hemostasis valve 156, hemostasis valve 334 may help to prevent blood from leaking out of the loader sheath 300 when it is inserted into the introducer 150. In some examples, the hemostasis valve 334 may be a duckbill valve, a cross-slit valve, or an adjustable valve such as a mechanically adjustable valve or a valve which may have its size adjusted by filling a bladder that at least partially forms the valve. It should be understood that, although hemostasis valve 334 is only shown in FIG. 12D, the other examples of loader sheaths described herein may in some examples similarly include a hemostasis valve. Further, in some examples, the cap 332 of tube 330 may be reversible coupled to the proximal hub 310 of the loader sheath 300. For example, the tube 330 may be coupled to the proximal hub 310 may screwing the proximal hub 310 onto and/or over the cap 332 of the tube 330.

It should be understood that, in the view of FIG. 12D, other components of the delivery system 100, such as the inner catheter 134, outer catheter 132, and steering catheter 135 are omitted from the view for ease of illustration and to more clearly show an example of the relationship between loader sheath 300 and introducer 150.

Whether or not loader sheath 300 is configured to telescope into the introducer 150, in some examples, if the loader sheath 300 is taking up too much space and the user needs to increase the working length of the delivery catheter 130 after the prosthetic valve 10 has passed distally to the hemostasis valve 156, the user may want to remove part of all of the loader sheath 300 from the delivery system 100. However, in some examples the loader sheath 300 fully surrounds a portion of the delivery catheter 130, and cannot be removed distally because the distal end of the assembled system is within the patient's vasculature, and cannot be removed proximally because the loader sheath 300 is too small to be removed over other proximal components (e.g., the handle 110) of the delivery system 100. Thus, if the loader sheath 300 is to be removed, in some examples, it must be cut longitudinally (e.g., after the proximal hub 310 is unscrewed from the cap 332 and moved out of the way) to allow the removal of the loader tube 330 to increase available working length of the delivery catheter 130. However, in some examples it may be relatively complex and/or time-consuming to perform such a procedure. In other examples, loader tube 330 may be formed of a “peelable” material such as FEP or PTFE. If loader tube 330 is formed of such a material, in some examples, a use may manually tear the tube 330 to allow it to be removed from over the delivery catheter 130, if desired. However, such a configuration may lead to one or more problems. For example, FEP and PTFE typically cannot be overmolded onto other components. Thus, for example if the tube 330 were to be formed of peelable FEP but the proximal cap 332 were formed of a separate (e.g., more rigid) material more suitable to threadedly connecting to proximal hub 310, the tube 330 may not be able to be overmolded onto the proximal cap 332, meaning another mechanism of connection, such as adhesives, may be necessary. However, coupling tube 330 to proximal cap 332 via adhesive may be generally undesirable because the loader sheath 300 may become more likely to leak across the point of connection. Still further, in some examples in which tube 330 is formed of a peelable material such as FEP, two perforations or other features may be provided to assist in initiating the step of peeling the tube 330 for removal from the delivery catheter 130. However, a known problem with such configurations is that, as material tears along the two tear lines, there may be a tendency for the tear lines to converge to a single point, making further manual tearing difficult. In those cases, it may be necessary to use a separate tool to cut the remainder of the tube 330, which may generally defeat the purpose of having tear-away features in the first place.

Referring now in addition to FIGS. 12E-G, FIGS. 12E-G are each schematic views of a transverse cross-section of a tube of a loader sheath formed from a material compatible with overmolding (such as polymers in the amide family including high durometer polyether block amides offered under the tradename Pebax®, clear nylon), which include features to assist with tearing the tube if necessary. For example, FIGS. 12E-G each show a transverse cross-section of a tube 330′, 330″, 330″′, respectively, of a loader sheath that may be identical to loader sheath 300, with the exception of the features that promote tearing. In the example of FIG. 12E, two longitudinally extending recesses or channels 331′ have been formed along tube 300′, with the two channels 331′ interrupting the otherwise substantially circular surface of the inner diameter ID (which may create weakened areas) while leaving a smooth outer diameter OD. In the example of FIG. 12F, two longitudinally extending recesses or channels 331″ have been formed along tube 300″, with the two channels 331″ interrupting the otherwise substantially circular surface of the outer diameter OD (which may create weakened areas) while leaving a smooth inner diameter ID. In the example of FIG. 12G, two longitudinally extending recesses or channels 331′″ have been formed along tube 300″′ entirely within the wall of the tube 300′″ (which may create weakened areas), with the two channels 331″′ being enclosed within the wall thickness to leave a substantially smooth inner diameter ID and outer diameter OD. In each example shown in FIGS. 12E-G, the tube may be overmolded onto the proximal cap (e.g., proximal cap 332) so that a complete effective seal is achieved between the two components. Further, by forming the tubes of the examples of FIGS. 12E-G of a material that is capable of overmolding, the resulting tube may be transparent or otherwise translucent, allowing for good visualization of components within the tube. Although the tubes of the examples of FIGS. 12E-G are not formed of a material like FEP which may be easily peelable, the cut-out or recessed channels may weaken the structural integrity of the tube enough to allow for peeling the tube along the channels, although enough structural integrity remains to for the loader sheath to perform its desired function.

Referring now in addition to FIG. 12H, FIG. 12H is a schematic view of a transverse cross-section of a tube 330″″ of a loader sheath. Tube 330″″ may be similar or identical to the tubes of FIGS. 12E-G, with the main exception that, in the example of FIG. 12H, instead of including two longitudinally extending channels or recesses, the tube 330″″ may be formed as a co-extrusion with two longitudinally extending strips of a soft, peelable polymer (such as low durometer polyether block amides offered under the tradename Pebax® or other material) 331″″ (which may create weakened areas), while the remainder of the tube 330″″ is formed of a more rigid polymer (or other material). Similar to tube 330″″ of FIG. 12G, tube 330″″ of FIG. 12H may include a substantially circular and/or smooth outer diameter OD and inner diameter ID, which may be desirable. Otherwise, tube 330″″ may function in the same way as the tubes of FIGS. 12E-G, with preferential zones being provided to allow for the tube 330″″ to be torn and then removed from the delivery catheter 130 if desired.

Referring now in addition to FIG. 12I, FIG. 12I is a schematic illustration of the tube 330 of loader sheath 300 which has been formed with two longitudinally extending peel-away channels 331. It should be understood that the tube 330 may be any of those shown and described in connection with FIGS. 12E-H, or any other tube of a loader sheath formed with longitudinally extending peel-away channels. In the example of FIG. 121, a peeling tab 333 is formed on an end of the tube 330 connected to both channels 331, for example via overmolding. The tab 333, if included, may be formed on either end of the tube 330, including the distal end of the proximal end. If the use decides additional working length is needed, or otherwise decides that the tube 330 of the loader sheath 300 should be removed from the delivery catheter 130, the user may grip the tab 333 and pull the tab 333 in the direction of the extend of the channels 331 to tear the tube 330. In the example of FIG. 12I, as well as the examples of FIGS. 12E-H, the inclusion of two longitudinally extending channels 331 (which may be parallel) may assist with the tearing occurring in a well-defined and predictable manner. For example, as shown in FIG. 12J, after completing the peeling by pulling tab 333, the tube 330 has ripped away along the channels 331, leaving a well-defined slit 335 extending the length of the tube 330. Once the slit 335 is formed, the tube 330 may be readily removed from the delivery catheter 130 to increase the working length of the delivery catheter 130. In examples in which two parallel longitudinally extending channels 331 are not provided to guide the tearing, and a user attempts to peel the tube (e.g., if it were formed of FEP), the tears may converge, forming a converging slit CS, as shown in the example of FIG. 12K. If a converging slit CS results, the user continuous outer diameter of the tube has not been broken along its entire length, and thus the user may need to manually cut away the remainder of the tube, effectively eliminating any benefit of the peel-away functionality. The embodiments described in connection with FIGS. 12E-J may, in some examples, avoid the likelihood of a converging slit CS resulting from a peel-away process.

Now referring in addition to FIGS. 12L-12M, FIG. 12L is a side view showing an example of loader sheath 300 in isolation and FIG. 12M is a longitudinal cross-section showing the loader sheath 300 assembled to an example distal end of a delivery system. FIGS. 12L-12M include examples of additional details of loader sheath 300, including showing an interaction between loader tube 330 being received within and coupled to cap 332. In some examples, the cap 332 may include external threading that threads into internal threading of a connector portion 310a of proximal hub 310. In some examples, an end cap 310b may snap onto (or otherwise couple to) the proximal end of the connector portion 310a. As shown in FIG. 12M, in some examples, hemostasis valve 334 may be provided sandwiched between the end cap 310b and the connector 310a of the proximal hub 310. It should be understood that the configuration of components of loader sheath 300 shown in FIGS. 12L-12M may be used, with or without modification, in any of the other loader sheaths described herein.

Referring again to FIG. 12L, in some examples, the loader tube 330 may have a length LT1 that extends distally to the cap 332 which may be available to enter into introducer 150. In some examples, the length LT1 may be between about 1.75 inches and about 2.25 inches, including about 2 inches. This length LT1 may be short relative to other devices, which may allow for between about 50% (+/−5%) and about 80% (+/−5%) of the length LT1 to telescope into the introducer 150, which may include about 60% (+/−5%) telescoping or about 70% (+/−5%) telescoping. In some examples, between about 1.1 inches and about 1.3 inches, including about 1.2 inches of the length LT1 is capable of telescoping into the introducer 150. The entire length LT2 of the tube 330 and cap 332 which extends distally of the proximal hub 310 may, in some examples, be between about 2.4 inches and about 2.7 inches, including about 2.5 inches, about 2.55 inches, or about 2.6 inches. In some examples, the length PHI of the proximal hub 310 may be between about 0.5 inches and about 0.75 inches, including about 0.55 inches, about 0.6 inches, about 0.65 inches, or about 0.7 inches.

Referring again to FIG. 12M, in some examples, when the loader sheath 300 is positioned near its fully distal position relative to a distal end of the delivery device, the distal end of tube 330 at least partially surrounds distal pillow 136b while the distal end of the steering sheath 135 is positioned at least slightly distal to hemostasis valve 334. It should be understood that, when fully assembled, the loader sheath 300 may be positioned slightly more distally relative to the delivery device components (e.g., to the left in the view of FIG. 12M) than is shown in FIG. 12M. For example, the proximal hub 310 may include an interior distal extension 310c that is configured to contact a proximal end of proximal pillow 136a when the loader sheath 300 is fully advanced, and the interior distal extension 310c may serve as a “hard stop” to prevent further distal movement of the loader sheath 300 relative to the delivery device.

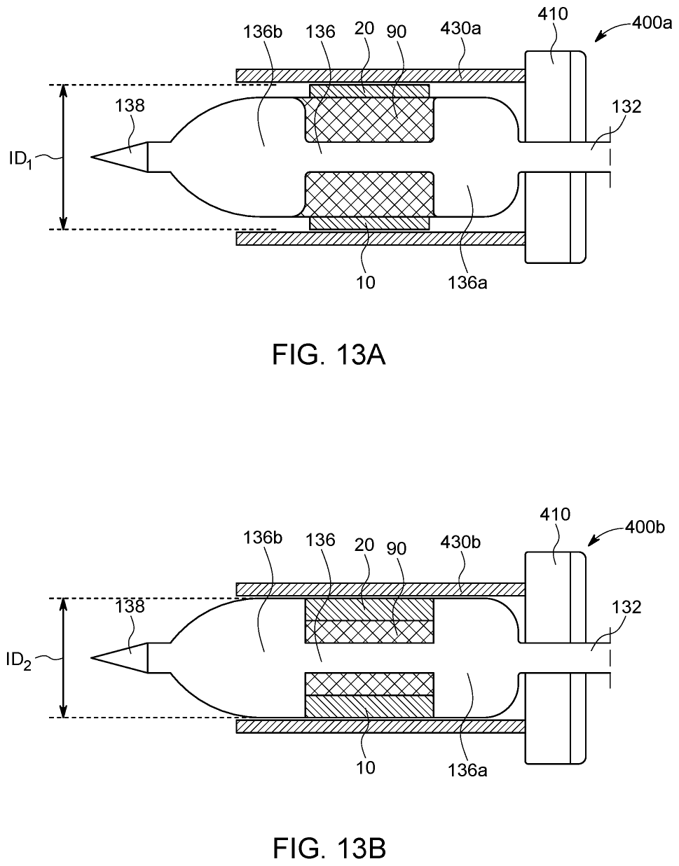

Now referring in addition to FIGS. 13A-13B, FIGS. 13A-13B illustrate highly schematic longitudinal cross-sections of a set of loader sheaths 400a, 400b. In one example, loader sheath 400a (shown in FIG. 13A) includes a proximal hub 410 and a tube 430a with a relatively large inner diameter ID₁, while loader sheath 400b (shown in FIG. 12B) includes the same proximal hub 410 but a different tube 430b, with a relatively small inner diameter ID₂. For example, loader tube 430a (which in some examples may include a proximal cap and/or hemostasis valve similar to that shown in FIG. 12D) may be removably couplable to the proximal hub 410, for example via a threaded connection that allows for the loader tube 430a to be screwed into or out of the proximal hub 410. Similarly, loader tube 430b (which in some examples may include a proximal cap and/or hemostasis valve similar to that shown in FIG. 12D) may be removably couplable to the proximal hub 410, for example via a threaded connection that allows for the loader tube 430b to be screwed into or out of the proximal hub 410. In other words, in some examples, a set of loader sheaths 400a, 400b may include a single proximal hub 410 but two loader tubes 430a, 430b of different sizes that may each be compatible with the single proximal hub 410.

In one example, the prosthetic heart valve 10 may be crimped over balloon 136 in a first stage of preparation, and loader sheath 400a, assembled to the delivery catheter 130 so that the tube 430a overlies the crimped prosthetic heart valve 10. This first stage of preparation in some examples may be performed by a manufacturer of the delivery system 100, or otherwise be performed at a time that is hours, days, weeks or more earlier than the intended use of prosthetic heart valve 10. In the illustrated example, this first stage of preparation results in the prosthetic heart valve 10 having a crimp profile (e.g., an outer diameter while crimped over balloon 136) that is relatively large, including larger than the outer diameter of the proximal pillow 136a of the balloon 136 and/or the distal pill of the balloon 136. The outer diameter of the prosthetic heart valve 10 in this configuration may, in some examples, be about equal to or slightly smaller than the inner diameter ID₁ of the tube 430a. After achieving the configuration of FIG. 13A, the delivery system 100, which may include the loader sheath 400a overlying the prosthetic heart valve 10 on the balloon 136, may be packaged for eventual sale and/or delivery to an end user. Because in this example the prosthetic heart valve 10 has been crimped but there is not an intent for immediate use of the prosthetic heart valve 10, it may be desirable to allow the prosthetic heart valve 10 to rest or otherwise remain at the relatively large inner diameter ID₁ within the tube 430a, as less stress and/or strain may be experienced by the prosthetic heart valve 10 during storage if it is maintained with a relatively large crimp profile.

After the end user receives and is ready to implant the prosthetic heart valve 10, it may be removed from the packaging while loader sheath 400a overlies the prosthetic heart valve 10. During a second stage of preparation, which may be performed immediately prior to the intended implantation of prosthetic heart valve 10, the end user (and/or personnel associated therewith) may in some examples unscrew the tube 430a from the proximal hub 410, and remove the tube 430a distally (e.g., in the direction to the left in the view of FIGS. 13A-13B) off of the distal end of the delivery catheter 130. At this point, the prosthetic heart valve 10 in some examples may have a crimp profile that is too large for use, and the user may insert the prosthetic heart valve 10 (while it is maintained on the balloon 136) in a crimping device to further crimp the prosthetic heart valve 10 to a smaller outer diameter/crimp profile. During this second stage of crimping, in some examples, the axial position of the prosthetic heart valve 10 is readily maintained due to the inflow end of the prosthetic heart valve 10 abutting distal pillow 136b and the outflow end of the prosthetic heart valve 10 abutting proximal pillow 136a. After the prosthetic heart valve 10 has been crimped to a smaller diameter (e.g., a smaller diameter than the inner diameter D₁ of the tube 430a) desirable for use. Then, in some examples, tube 430b, which may be provided within or as part of the packaging for delivery system 100, may be slid over the balloon 136 and the prosthetic heart valve 10, and screwed into or otherwise connected to proximal hub 410. As shown in FIG. 13B, in some examples, the tube 430b may have an inner diameter ID₂ that is smaller than the inner diameter ID₁ of tube 430a, and inner diameter ID₂ may be about equal (or slightly larger) than the outer diameter of the prosthetic heart valve 10 when the prosthetic heart valve 10 is in a crimped state suitable for implantation into the patient.

As should be understood from the above description, in some examples, the use of two loader sheaths 400a, 400b may provide the benefit of having a loader tube protecting the prosthetic heart valve 10 between the initial time point of initial assembly and/or packaging, and the later time point of the actual implantation of the prosthetic heart valve 10. However, by including a relatively large tube 430a and a relatively small tube 430b that are both compatible with proximal hub 410, the initial longer term storage of the prosthetic heart valve 10 may be achieved while the prosthetic heart valve 10 is in a larger crimp profile and thus in some examples subjected to less stress and/or strain. And the relatively small tube 430b, in some examples, may be used just prior to implantation to achieve any or all of the benefits described with other loader sheaths herein, other than protection during packaging, which may be provided by the larger tube 430a. It should be understood that, in examples, loader sheath 400b may include features of other loader sheaths described herein, including for example features that assist with peeling, such as those shown and described in connection With FIGS. 12A-12J. In some examples, the first loader tube 430a is neither sized for being advanced into the introducer 150 nor is the first loader tube 430a intended to cover the prosthetic heart valve 10 while the prosthetic heart valve 10 is being delivered into the patient.

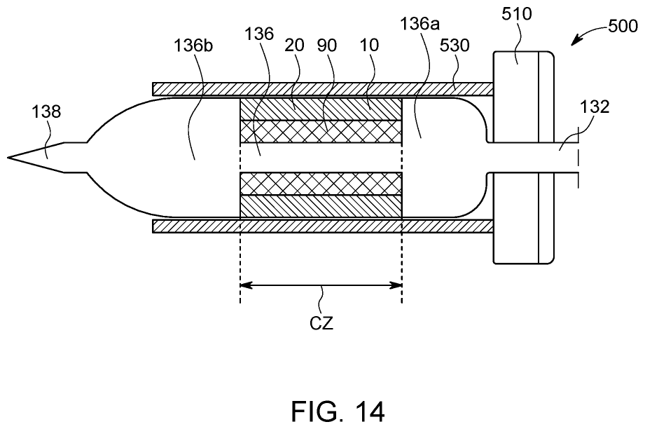

Now referring in addition to FIG. 14, FIG. 14 is a highly schematic longitudinal cross-section of an example of a loader sheath 500 assembled over prosthetic heart valve 10 which is crimped over balloon 136 of the delivery system 100. Whereas other examples of loader sheaths described herein may be formed mostly or entirely of a relatively rigid material such as FEP, PTFE, nylon, or HDPE, at least a portion of the tube 530 of loader sheath 500 may be formed of a semi-compliant material, such as low durometer Pebax®. In some examples, the entire length of tube 530 may be formed of the semi-compliant material, while in other examples, an intermediate length of the tube 530, which may be referred to as crimp zone CZ, may be formed of the semi-compliant material, while the portions of tube 530 distal to and/or proximal to the crimp zone CZ are formed of the relatively rigid and/or non-compliant material. In some examples, the crimp zone CZ of the tube 530 may have a length that is about equal to a length of the balloon 136 between the proximal pillowed portion 136a and the distal pillowed portion 136b, and/or about equal to a length of the prosthetic heart valve 10 when the prosthetic heart valve is in a crimped condition. With this configuration, in some examples, the prosthetic heart valve 10 may be crimped over balloon 136 in a first crimping step (e.g., performed by the manufacturer or by the end user), and the loader sheath 500 may be placed over the crimped prosthetic heart valve 10 so that the crimp zone CZ of the tube 530 is axially aligned with the axial length of the prosthetic heart valve 10. Then, in some examples, during a second crimping step (e.g., performed by the manufacturer or the end user after the first crimping step), a crimping device, such as a manual crimper, may be placed over the stack-up of the prosthetic heart valve 10 and the crimp zone CA of the tube 530, and a second crimping may be performed to further decrease the size of the prosthetic heart valve 10. In these examples, because the crimp zone CZ is semi-compliant, the crimper is capable of further crimping the prosthetic heart valve 10 while it is inside the tube 530, which may not be feasible if the crimp zone CA were formed from a relatively rigid material. In some examples, this configuration may allow for the prosthetic heart valve 10 to be stored with a relatively large crimp profile, which may reduce potential damage to the prosthetic heart valve 10 while it is in storage before use.

In some examples of loader sheath 500, a single loader sheath may be used during the manufacture and initial packaging of delivery system 100, and that seme loader 500 may remain over the prosthetic heart valve 10 during additional downstream crimping (e.g., just prior to implantation of the prosthetic heart valve) and may remain on the delivery catheter 130 at least up until the point at which the prosthetic heart valve 10 has been passed into the introducer 150. In other examples, loader sheath 500 may be used for crimping steps but may be removed prior to implantation of the prosthetic heart valve 10. For example, loader sheath 500 may be provided over the prosthetic heart valve 10 while the delivery system 100 is packaged, and if desired, an end user may perform a crimping procedure over the loader sheath 500 prior to implantation of the prosthetic heart valve 10 in substantially the same manner as described above. But, in this example, the loader tube 530 may be unscrewed from the proximal hub 510 (much like tube 430a of FIG. 13A is unscrewed from proximal hub 410), and a more rigid, smaller diameter tube, such as tube 430b of FIG. 13B, may then be placed over prosthetic heart valve 10 and coupled to proximal hub 510. In such examples, while the smaller diameter, more rigid tube 430b is coupled to proximal hub 510, de-airing may be performed, and the tube 430b may be coupled to proximal hub 510 for use during implantation of the prosthetic heart valve 10. Further, in some examples, tube 530 may include features of other delivery sheath tubes described herein, such as the peel-away features of the tubs of FIGS. 12E-12J.

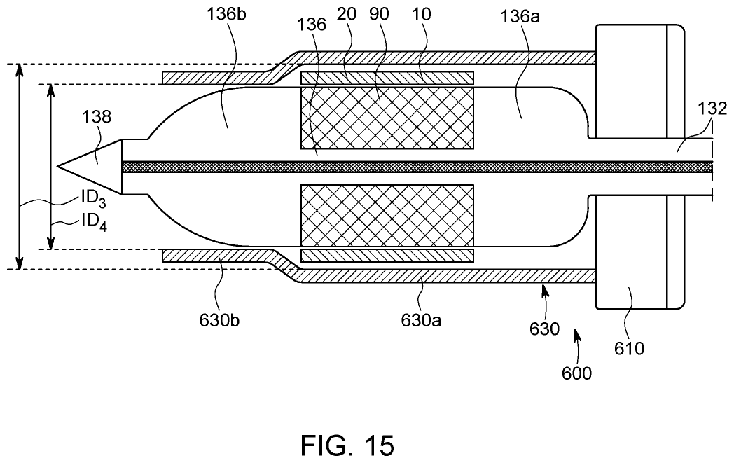

Now referring in addition to FIG. 15, FIG. 15 is a highly schematic longitudinal cross-section of an example of a loader sheath 600 assembled over prosthetic heart valve 10 which is crimped over balloon 136 of the delivery system 100. Whereas other examples of loader sheaths described herein may include tubes with substantially constant inner diameters, in the illustrated example, the tube 630 of loader sheath 600 may include sections with different diameters. In the illustrated example, the tube 630 may include a proximal section 630a that has a proximal end configured to couple (e.g., via screw threads) to the proximal hub 610, and a distal end configured to overlie the inflow end of the prosthetic heart valve 10 when the prosthetic heart valve 10 is crimped over the balloon 136. In some examples, the proximal section 630a of tube 630 may have a length that is about equal to or slightly larger than the combined axial length of the proximal pillowed portion 136a of the balloon 136, and the central portion of the balloon 136 over which prosthetic heart valve 10 is crimped. In some examples, this proximal section 630a has a substantially constant inner diameter ID₃, which may have size generally similar to inner diameter ID₁ of FIG. 13A. In other words, in some examples, inner diameter ID₃ is larger than the desired size of the crimp profile of the prosthetic heart valve 10 during delivery of the prosthetic heart valve 10. For example, in some examples, a manufacturer my crimp the prosthetic heart valve 10 to have a crimp profile that is about equal to inner diameter ID₃, and the loader sheath 600 may protect the prosthetic heart valve 10 during storage and/or delivery, with the prosthetic heart valve 10 remaining within the proximal section 630a. As with loader sheath 400a, in some examples, loader sheath 600 may allow the prosthetic heart valve 10 to be relatively large prior to use, even though it is still crimped over balloon 136, to reduce the amount of stress and/or strain on the prosthetic heart valve 10 prior to use.

In some examples, the tube 630 of loader sheath 600 may include a distal section 630b extending from the distal end of proximal section 630a, the distal section 630b decreasing in inner diameter in a tapered fashion between the connection to proximal section 630 to the distalmost end of the distal section 630b, where the distal tube 630b has an inner diameter ID₄ that is smaller than inner diameter ID₃. In some examples, the taper may be substantially uniform, although in other examples the taper may be non-uniform, with an initial large reduction in diameter adjacent to the distal end of proximal section 630a, and then a more gradual reduction in diameter distal to the initial large reduction in diameter. In some examples, inner diameter ID₄ may be about equal to the final desired crimp profile of the prosthetic heart valve 10 when it is being advanced through the vasculature, which may be similar in some example to inner diameter ID₂ shown in FIG. 13B, and/or the inner diameter of tube 330 of FIG. 12A. With the configuration of the example of FIG. 15, in some examples, the prosthetic heart valve 10 may be crimped over balloon 136 in a first crimping step (e.g., performed by the manufacturer or by the end user), and the loader sheath 600 may be placed over the crimped prosthetic heart valve 10 so that the prosthetic heart valve 10 may be stored within loader sheath 600 with a relatively small amount of stress and/or strain prior to use. Then, in some examples, the end user may prepare the delivery system 100 without needing to remove loader sheath 600. During use, in some examples, the distal end of the loader sheath 600 will eventually contact or lock to the introducer 150, for example similar to the configuration shown in FIG. 12D. In this example, the loader sheath 600 stops translating relative to the introducer 150, but the delivery catheter 130, with the prosthetic heart valve 10 crimped thereon, may continue to be passed into and through the introducer 150. As this occurs, in some examples, the prosthetic heart valve 10 will translate distally relative to the tube 630, eventually passing into and through the distal section 630b. The distal section 630b, in some examples, acts as a funnel and forces the prosthetic heart valve 10 to further crimp to a smaller profile over the balloon 136 as the prosthetic heart valve passes distally through the distal section 630b of the tube 630. In this example, after the prosthetic heart valve 10 is further crimped to the smaller profile after exiting the loader sheath 600, it may have the desired crimp profile for the remainder of the procedure while the prosthetic heart valve 10 traverses the patient's vasculature.

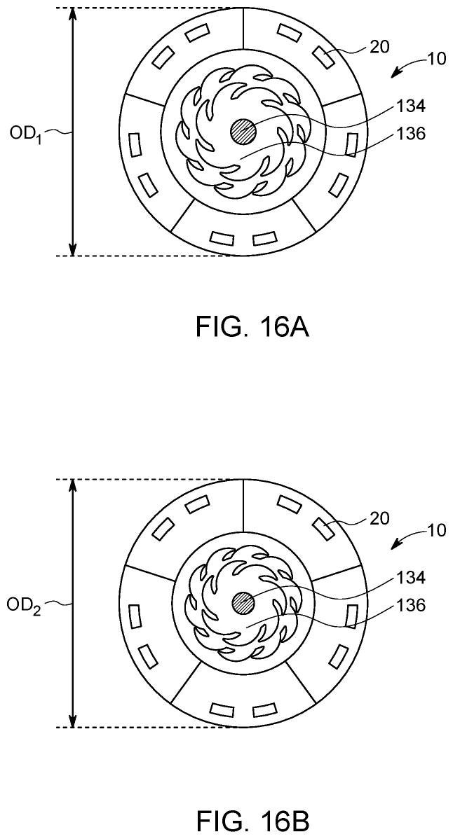

Referring now in addition to FIG. 16A, FIG. 16A is a highly schematic transverse cross section of an example of prosthetic heart valve 10 crimped over balloon 136 and inner catheter 134. When the prosthetic heart valve 10 is in the crimped condition, for example if crimping is performed by the manufacturer before packaging and/or by the user immediately prior to implantation of the prosthetic heart valve 10, the prosthetic heart valve 10 may have a crimp profile or outer diameter OD₁. However, it may be desirable to reduce the size of the prosthetic heart valve 10 further to achieve an even smaller crimp profile for delivery of the prosthetic heart valve 10 through the patient's vasculature. In some examples, it may not be possible or practical to simply crimper the prosthetic heart valve 10 to a smaller outer diameter by merely applying more force when using the crimping device. However, in some examples, when the crimping is performed (e.g., by the manufacturer prior to packaging and/or by the end user immediately prior to implantation of the prosthetic heart valve 10), a vacuum may be pulled within balloon 136. It should be understood that, as used herein, the phrase “pulling a vacuum” does not necessarily require a true vacuum be achieved, but rather references a process in which pressure within a system is decreased. For example, syringe 174 (or another syringe) may be fluidly coupled to the balloon 136, and the plunger handle 182 of the syringe 174 (or other syringe) may be pulled proximally, including either manually or with the assistance of balloon inflation system 170, to withdraw as much possible fluid (e.g., air or saline) that otherwise remains in balloon 136. By crimping the prosthetic heart valve 10 over the balloon 136 while all the fluid (or most of the fluid) within balloon 136 has been withdrawn by creating a vacuum (or otherwise creating a negative pressure differential), the combination of the prosthetic heart valve 10 and balloon 136 may be crimped to a smaller diameter than if more fluid remained within the balloon 136. As shown in the example of FIG. 16B, an outer diameter OD₂ or crimp profile of the prosthetic heart valve 10 may be achieved, with outer diameter OD₂ being smaller than outer diameter OD₁, because most or all of the fluid previously within the balloon 136 has been displaced from the balloon 136. In some examples, if crimping is performed while pulling a vacuum, after the crimping, any of the loader sheaths described herein may be placed over the prosthetic heart valve 10 to help the prosthetic heart valve 10 maintain the smaller crimp profile OD₂ for implantation.

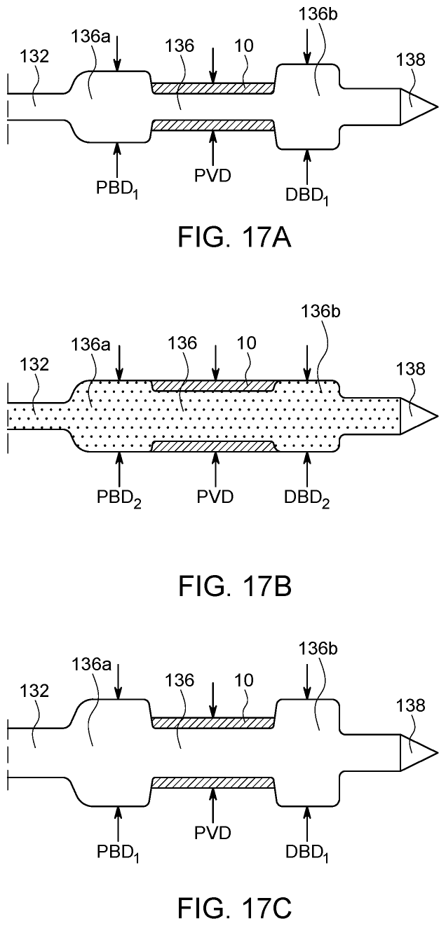

As noted above, in some examples, vacuum pressure may be utilized to decrease the crimped profile of the prosthetic heart valve 10. In other examples, vacuum pressure may be utilized to manipulate the size of the balloon itself during delivery of the prosthetic heart valve. For example, referring now in addition to FIG. 17A, FIG. 17A is a highly longitudinal cross-section of the distal end of delivery catheter 130, with the prosthetic heart valve 10 crimped over balloon 136 and the axial ends of the prosthetic heart valve 10 contacting the proximal pillow 136a and distal pillow 136b of the balloon 136. In this example, the proximal pillow 136a has a proximal balloon diameter PBD₁ and the distal pillow 136b has a distal balloon diameter DBD₁, with the prosthetic heart valve 10 having a crimp profile or a prosthetic valve diameter PVD. In some examples, the proximal balloon diameter PBD₁ and the distal balloon diameter DBD₁ may each be larger than the prosthetic valve diameter PVD, and in some examples the proximal balloon diameter PBD₁ and the distal balloon diameter DBD₁ may each be about equal. In one particular example, the proximal balloon diameter PBD₁ and the distal balloon diameter DBD₁ may each be about 10 mm (e.g., between about 8 mm and about 12 mm), while the prosthetic valve diameter PVD may be about 8 mm (e.g., between about 6 mm and about 10 mm).

Now referring in addition to FIG. 17B, FIG. 17B illustrates the configuration of FIG. 17A after a vacuum has been pulled to withdraw most or all of the remaining fluid within balloon 136. As a result of fluid being withdrawn, in some examples the relatively large profiles of the proximal pillow 136a and distal pillow 136b may reduce. In one example, after pulling a vacuum within balloon 136, the proximal pillow 136a may reduce to a proximal balloon diameter PBD₂ and the distal pillow 136b may reduce to a distal balloon diameter DBD₂, which in some examples may be equal to each other and to the prosthetic valve diameter PVD. In one example, after pulling the vacuum, the proximal balloon diameter PBD₂, the distal balloon diameter DBD₂, and the prosthetic valve diameter PVD, may all be about 8 mm (e.g., between about 6 mm and about 10 mm). In one example, the vacuum may be pulled to shift the balloon 136 to the configuration shown in FIG. 17B (or a similar configuration) just prior to insertion of the prosthetic heart valve 10 into the introducer 150 and through the vasculature. As the delivery catheter 130 is advanced further through the patient's vasculature, which may be a femoral artery if the prosthetic heart valve 10 is a prosthetic aortic valve, the vacuum may be maintained to keep the balloon 136 in the configuration shown in FIG. 17B (or a similar configuration). At some point during the delivery at which it becomes desirable for the proximal pillow 136a and/or distal pillow 136b to have an increased diameter relative to the crimped prosthetic heart valve 10, the vacuum may be released so that, as shown in the example of FIG. 17C, the proximal balloon 136a and distal balloon 136b return to the original configuration (e.g., back to the example configuration of FIG. 17A prior to insertion into the patient). In one example, when prosthetic heart valve 10 is a prosthetic aortic valve being delivered via a transfemoral route, the vacuum may be released when the prosthetic heart valve 10 is within the descending aorta. With this example, the proximal pillow 136a and distal pillow 136b will return to their relatively larger diameter as the prosthetic heart valve 10 tracks around the aortic arch and into the native aortic valve annulus, which are both locations at which the protection provided by the larger proximal pillow 136a and distal pillow 136b may be most desirable. For example, as the prosthetic heart valve 10 curves around the aortic arch, and as the prosthetic heart valve approaches and enters the native aortic valve annulus, the irks may be relatively high for a leading edge of the prosthetic heart valve 10 (e.g., the outflow end) to contacting native tissue and dislodge the prosthetic heart valve 10 from its position on balloon 136. However, in some examples, if the proximal pillow 136a and the distal pillow 136b have been returned to their larger diameter (e.g., by releasing a previously-pulled vacuum), the proximal pillow 136a and distal pillow 136b may provide protection to and increased retention of the prosthetic heart valve 10 around the aortic arch and within the native aortic valve annulus. The increased diameters of proximal pillow 136a and distal pillow 136b may also, in some examples, provide for enhanced valve retention as the balloon 136 is inflated for deployment of the prosthetic heart valve 10. At prior stages of delivery, the proximal pillow 136a and distal pillow 136b may have a smaller diameter due to a pulled vacuum, allowing for the balloon 136 to have an overall smaller profile during the earlier stages of delivery. In these examples, benefits of having a small diameter for the proximal pillow 136a and distal pillow 136b and benefits of having a large diameter for the proximal pillow 136a and distal pillow 136b may both be achieved within the same procedure.

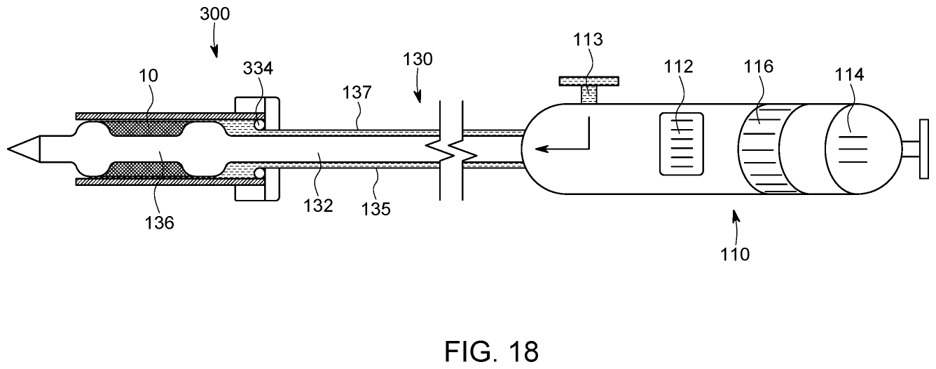

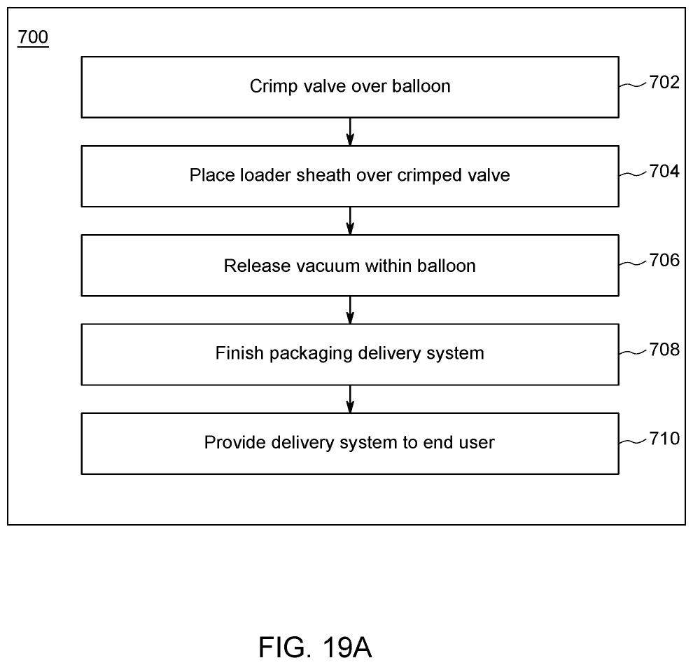

Now referring in addition to FIG. 18, FIG. 18 is a highly schematic illustration of handle 10 and delivery catheter 130, with prosthetic heart valve 10 crimped over balloon 136 and covered by loader sheath 300 (although it should be understood that the concepts described in connection with FIG. 18 may be equally applied to other examples of loader sheaths, including other examples described herein). Prior to using delivery system 100 to implant prosthetic heart valve 10, it is important in some examples to flush the various lumens, for example with saline to help remove any air from the system prior to inserting the delivery device into the patient. In the example illustrated in FIG. 18, a flush port 113 is provided on handle 110 which leads to the space between the outer diameter of the outer catheter 132 and the inner diameter of the steering catheter 135, which space may be referred to as a flush lumen 137. In the illustrated embodiment, when the loader sheath 300 is assembled over the prosthetic heart valve 10, the distal end of the steering catheter 135 may be aligned with the hemostasis valve 334 (e.g., the distal end of the steering catheter 135 may terminate just distal to the hemostasis valve 334) of the loader sheath 300. With this example configuration, a user may pass saline or other flushing fluid into the flush port 113, causing that saline or other flushing fluid to pass through the flush lumen 137 between the outer catheter 132 and the steering catheter 135, and also into the interior space of the loader sheath 300, allowing the steering catheter 135, the loader sheath 300, and the prosthetic heart valve 10 to be flushed simultaneously using only a single flush port 113. In some examples, the loader sheath 300 may be configured to automatically align for this flushing functionality when the loader sheath 300 is pushed fully distally by including a hard stop (e.g., interior distal extension 310c of FIG. 12M) within the loader sheath the contacts the proximal pillow 136a to restrict further advancement. In other words, with the example configurations above, the time require to prepare the delivery system 100 may be reduced compared to configurations that require separate flushing of the steering catheter 135 and the loader sheath 300 (including prosthetic heart valve 10). In addition, in these configurations, the number of flush line features in the delivery system 100 may be reduced compared to configurations that require separate flushing of the steering catheter 135 and the loader sheath 300 (including prosthetic heart valve 10). It should be noted that FIG. 12M illustrates an example positioning that may also provide for the continuous flushing as described in connection with FIG. 18.

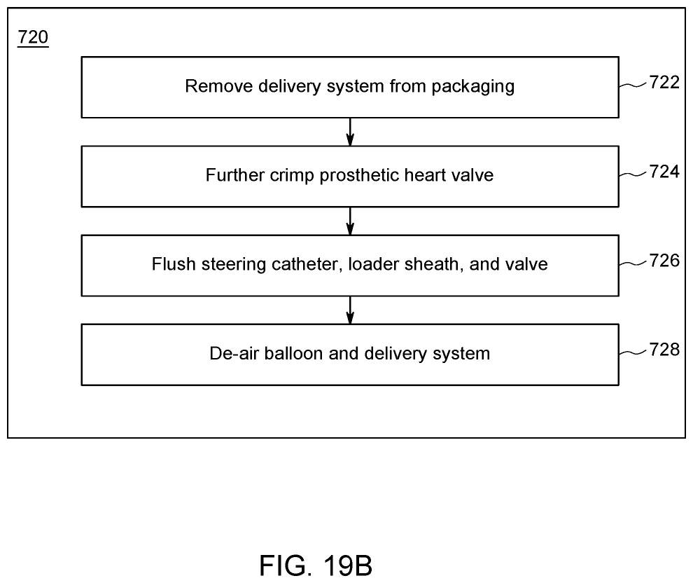

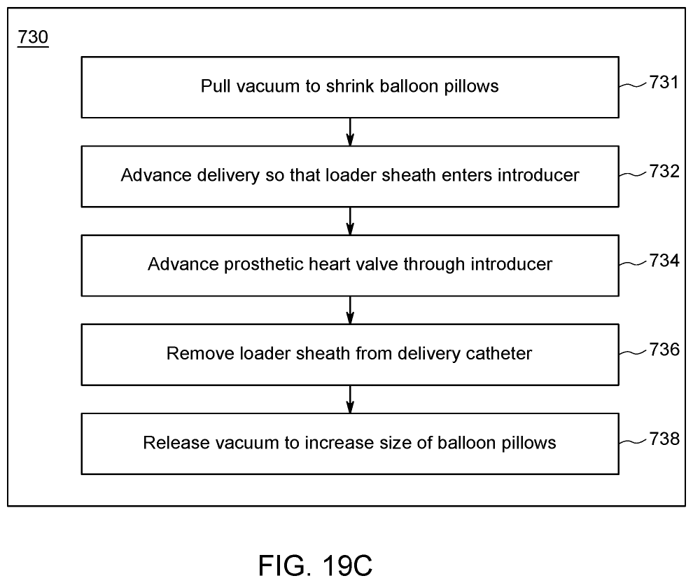

Now referring in addition to FIG. 19A, FIG. 19A is a flowchart of an example of a first stage of a method 700 of preparing a delivery system (e.g., delivery system 100) for implanting a prosthetic heart valve (e.g., prosthetic heart valve 10). In one example of a first step 702 of preparation, the prosthetic heart valve may be crimped over a balloon (e.g., balloon 136) of the delivery system. In some examples, step 702 may be performed by a manufacturer of the delivery system prior to providing the delivery system to an end user. In one example of a second stop 704 of preparation, a loader sheath may be placed over the crimped prosthetic heart valve. The loader sheath may be any desirable loader sheath. For example, step 704 may include placing loader sheath 300 over the prosthetic heart valve 10 to achieve the configuration of FIG. 12A. In another example, the loader sheath 300 used in step 704 may include any of the peel-away tubes of FIGS. 12E-12I. In another example, the loader sheath used in step 704 may be loader sheath 400a with a relatively large tube 430a, which may allow for the prosthetic heart valve 10 to be stored and/or packaged with relatively little stress and/or strain. In a further example, the loader sheath used in step 704 may be loader sheath 500, and the loader sheath 500 may be positioned so that the prosthetic heart valve is aligned with the crimp zone CZ of the tube 530 of loader sheath 500. In another example, the loader sheath used in step 704 may be loader sheath 600, and the prosthetic heart valve may be positioned within the proximal section 630a of tube 630, which may allow for the prosthetic heart valve 10 to be stored and/or packaged with relatively little stress and/or strain.

It should be understood that, in some examples, steps 702 and 704 may be performed while a vacuum is pulled on the balloon (or otherwise the pressure within the balloon is temporarily reduced), but in other examples, steps 702 and 704 may be performed without the use of pulling a vacuum. If steps 702 and 704 are performed while a vacuum is pulled within the balloon, as noted above, it may be possible to achieve a smaller crimp diameter of the prosthetic heart valve, for example similar to the configuration of FIG. 16B. If steps 702 and 704 are performed while a vacuum is pulled within the balloon, after placing the loader sheath over the crimped prosthetic heart valve in step 704, in some examples the vacuum may be released in step 706, with the loader sheath maintaining the prosthetic heart valve in the reduced-size crimped configuration.

Now referring in addition to FIG. 19B, FIG. 19B is a flowchart of an example of a second stage of a method 720 of preparing a delivery system (e.g., delivery system 100) for implanting a prosthetic heart valve (e.g., prosthetic heart valve 10). In some examples, method 700 may be performed by a manufacturer while method 720 may be performed by an end-user (alone or in combination with additional personnel associated with the end user). In one example, a first step 722 of method 720 is removing the assembled delivery system from the packaging. In some examples, a second step 724 of method 720 includes further crimping the prosthetic heart valve. In one example of step 724, if the delivery system was provided with loader sheath 400a, the tube 430a may be unscrewed from proximal hub 410 and removed, with the prosthetic heart valve crimped with a manual crimping device after the tube 430a is removed. It should be understood that, as part of step 724, in the example in which loader sheath 400a is provided with the delivery system, after the prosthetic heart valve is further crimped, another tube such as tube 430b may be placed over the further-crimped prosthetic heart valve and the tube 430b may be coupled (e.g., screwed into or onto) the proximal hub 41. In another example of step 724, if the delivery system was provided with loader sheath 500, a manual crimper may be placed over the crimp zone CZ of the tube 530 of the loader sheath 500, and the prosthetic heart valve crimped further while tube 530 remains covering the prosthetic heart valve. It should be understood that, in examples in which step 724 is performed, it may be performed while a vacuum is being pulled, similar to as described in connection with method 700. It should also be understood that, in some examples in which the loader sheath provided in step 700 is intended for use in the actual implantation procedure, step 724 may be omitted.

After the prosthetic heart valve is crimped to the size suitable for delivery and the loader sheath is over the prosthetic heart valve maintaining it in the desired crimped size, in some examples flushing and de-airing may be performed to further prepare the delivery system for use. In some examples, third step 726 may include simultaneously flushing a steering catheter (e.g., steering catheter 135), the loader sheath, and the prosthetic heart valve, including for example using saline. This may be accomplished, in one example, using the configuration of FIG. 18, or a similar configuration. In some examples, fourth step 728 may include de-airing the balloon as well as the fluid line(s) in communication with the balloon. As part of de-airing step 728, in some examples, the balloon may be pressurized while the loader sheath overlies the prosthetic heart valve, for example to help compress any air bubbles within the balloon and to help make it easier for such air bubbles to be withdrawn from the balloon. In some examples, the balloon inflation system (e.g., balloon inflation system 170) may be utilized to help automate the de-airing process, for example including using one or more methods described in U.S. Patent Application Publication No. 2023/0372097, the disclosure of which is hereby incorporated by reference herein. It should be understood that flushing step 726 and de-airing step 728, if performed, may be performed in an order other than as specifically shown in FIG. 19A.

Now referring in addition to FIG. 19C, FIG. 19C is a flowchart of an example of a method 730 of delivering a prosthetic heart valve (e.g., prosthetic heart valve 10) into a patient using a delivery system (e.g., delivery system 100). Once the delivery system has been prepared for use, entry into the patient's vasculature may be gained, for example using a guidewire similar to that described in connection with step 204 of method 200, and then advancing an introducer (e.g., expandable introducer 150) into the patient over the guidewire. Prior to starting to advance the prosthetic heart valve into the introducer, in some examples, an optional first step 731 may be performed in which a vacuum is pulled on the balloon (e.g., balloon 136) to shrink the proximal pillow (e.g., proximal pillow 136a) and/or the distal pillow (e.g., distal pillow 136b), for example to achieve the configuration shown in FIG. 17B. Once the introducer is within the patient, the user may in an example second step 732 advance the delivery system over the guidewire until the loader sheath (e.g., loader sheath 300) has passed through the hemostasis valve (e.g., hemostasis valve 156) of the introducer. In some examples of second step 732, locking features of the loader sheath (e.g., couplers 340) may be locked to corresponding locking features of the introducer (e.g., couplers 158) so that the loader sheath and the introducer effectively become a single unit, for example similar to the configuration shown in FIG. 12D. In an example of a third step 734, after the loader sheath has cleared the hemostasis valve of the introducer, the user may continue advancing the delivery device (e.g., delivery catheter 130) so that the prosthetic heart valve is advanced through the introducer sheath while the loader sheath no longer advances. As part of third step 734, in examples in which the loader sheath has a configuration similar to loader sheath 600, as the prosthetic heart valve is advanced through the loader sheath it may further crimp to a smaller size. After the prosthetic heart valve has been advanced out of the loader sheath, at any point, the loader sheath may be removed from the delivery catheter to increase the working length of the delivery catheter, if desired, as part of a fourth step 736. For example, if the loader sheath includes peel-away features, including any of those shown and described in connection with FIGS. 12E-12J, the loader sheath may be peeled to create a channel, and the loader sheath may be removed from the delivery catheter using that channel. However, it should be understood that fourth step 736 may be completely optional, and the loader sheath may in some examples remain attached to the delivery catheter for the remainder of the procedure. In an example of a fifth step 738, just prior to or after the prosthetic heart valve has reached the descending aorta (in the case in which the prosthetic heart valve is a prosthetic aortic valve being delivered via a transfemoral route), the vacuum pulled in step 731 (if step 731 was performed) may be released to allow the proximal pillow and/or distal pillow to “re-puff,” which may include allowing some amount of fluid to return into the proximal pillow and/or distal pillow. Although in this particular example, the vacuum release of step 738 is described as being performed when the prosthetic heart valve reaches the descending aorta, it should be understood that if the vacuum release is performed, it may be performed when the prosthetic heart valve is at another location within the patient's anatomy. Once step 738 is completed (if it is performed in the first place), the remainder of the prosthetic heart valve delivery and deployment may proceed, for example by performing one or more of steps 208, 210, 212, 214, 216, and 218 of method 200. It should further be understood that, although various individual steps are listed in a particular order in methods 700, 720, and 730, individual steps within the methods may be omitted as desired and/or performed in different orders than those explicitly shown if desired by the user.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A prosthetic heart valve delivery system comprising:
a delivery device having a balloon at a distal end thereof;
a balloon-expandable prosthetic heart valve configured to be received on the balloon in a crimped condition; and
a loader sheath configured to at least partially surround the crimped prosthetic heart valve while the crimped prosthetic heart valve is received on the balloon;
wherein the loader sheath includes a tube and a proximal hub, the loader sheath including a first channel extending in an axial direction along a wall of the tube and a second channel extending parallel to the first channel, the first channel and the second channel forming weakened areas to promote tearing of the tube along the first channel and along the second channel.

2. The prosthetic heart valve delivery system of claim 1, wherein the tube has an outer diameter and an inner diameter, and the first channel and the second channel are formed in a wall of the tube such that the first channel and second channel open to the inner diameter of the tube.

3. The prosthetic heart valve delivery system of claim 1, wherein the tube has an outer diameter and an inner diameter, and the first channel and the second channel are formed in a wall of the tube such that the first channel and second channel open to the outer diameter of the tube.

4. The prosthetic heart valve delivery system of claim 1, wherein the tube has an outer diameter and an inner diameter, and the first channel and the second channel are formed entirely within a wall thickness of the tube so that the first channel and the second channel are each enclosed within the wall thickness of the tube.

5. The prosthetic heart valve delivery system of claim 1, wherein the tube is overmolded on the proximal hub.

6. The prosthetic heart valve delivery system of claim 5, wherein the tube is formed of fluorinated ethylene propylene (“FEP”), polytetrafluoroethylene (“PTFE”), nylon, or high density polyethylene (“HDPE”).

7. The prosthetic heart valve delivery system of claim 1, wherein the first channel and the second channel are formed of a first polymer, and portions of the tube excluding the first channel and the second channel are formed of a second polymer, the second polymer having a rigidity that is greater than a rigidity of the first polymer.

8. The prosthetic heart valve delivery system of claim 1, further comprising a pull tab fixed to the tube, the pull tab directly connecting to the tube at locations of the first channel and the second channel.

9. A method of preparing a delivery system for an implantation, the method comprising:
coupling a fluid reservoir to a delivery device of the delivery system so that the fluid reservoir is in fluid communication with a balloon of the delivery device; and
while a balloon-expandable prosthetic heart valve is crimped over the balloon, and while a loader sheath at least partially surrounds the crimped prosthetic heart valve and the balloon, pressurizing the balloon by pressurizing the fluid reservoir,
wherein when pressurizing the balloon, the loader sheath limits an amount which the balloon and the prosthetic heart valve may expand.

10. The method of claim 9, wherein pressurizing the balloon causes at least one air bubble within the balloon to compress.

11. The method of claim 10, further comprising, after pressurizing the balloon, depressurizing the fluid reservoir to withdraw the at least one air bubble from the balloon.

12. A prosthetic heart valve delivery system comprising:
a delivery device having a balloon at a distal end thereof;
a balloon-expandable prosthetic heart valve configured to be received on the balloon in a crimped condition;
an introducer having an introducer hub and an introducer sheath extending distally from the introducer hub, a first hemostasis valve being positioned within the introducer hub; and
a loader sheath configured to at least partially surround the crimped prosthetic heart valve while the crimped prosthetic heart valve is received on the balloon, the loader sheath including a tube sized to be received within the introducer hub,
wherein the introducer includes a first coupler and the loader sheath includes a second coupler, the first coupler configured to couple to the second coupler when the tube of the loader sheath is received within introducer hub.

13. The prosthetic heart valve delivery system of claim 12, wherein the first coupler and the second coupler are each threads.

14. The prosthetic heart valve delivery system of claim 12, wherein the first coupler and the second coupler include a latch, hook, or detent.

15. The prosthetic heart valve delivery system of claim 12, wherein when the first coupler is coupled to the second coupler, the tube of the loader includes a distal length received within the introducer hub and a proximal length extending out of the introducer hub.

16. The prosthetic heart valve delivery system of claim 12, wherein when the first coupler is coupled to the second coupler, the distal length and the proximal length are each about half of a total length of the tube.

17. The prosthetic heart valve delivery system of claim 12, wherein when the first coupler is coupled to the second coupler, the distal length is about 75% of a total length of the tube and the proximal length is about 25% of the total length of the tube.

18. The prosthetic heart valve delivery system of claim 12, wherein when the first coupler is coupled to the second coupler, the distal length is about 25% of a total length of the tube and the proximal length is about 75% of the total length of the tube.
